(19) United States
(12) Patent Application Publication
(10) Pub. No.: US 2024/0016706 A1
(43) Pub. Date: Jan. 18, 2024

(54) MATERIALS FOR OBTURATION

(71) Applicant: SONENDO, INC., Laguna Hills, CA (US)

(72) Inventors: Zaza Gomurashvili, Tustin, CA (US); Ngoc Dinh Phuong Vo, Buena Park, CA (US); David Tobia, Laguna Hills, CA (US); Mehrzad Khakpour, Laguna Hills, CA (US); Bjarne Bergheim, Laguna Hills, CA (US)

(21) Appl. No.: 18/251,664

(22) PCT Filed: Nov. 2, 2021
(86) PCT No.: PCT/US2021/072194

§ 371 Date: May 3, 2023

Related U.S. Application Data
(60) Provisional application No. 63/109,794, filed on Nov. 4, 2020.
(60) Provisional application No. 63/109,797, filed on Nov. 4, 2020.
(60) Provisional application No. 63/109,802, filed on Nov. 4, 2020.
(60) Provisional application No. 63/115,493, filed on Nov. 18, 2020.
(60) Provisional application No. 63/247,709, filed on Sep. 23, 2021.

(51) Int. Cl. A61K 6/891; A61K 6/76; A61K 6/61; A61K 6/62; A61K 6/64; C08L 71/02

(52) U.S. Cl. CPC A61K 6/891; A61K 6/76; A61K 6/61; A61K 6/62; A61K 6/64; C08L 71/02

(57) ABSTRACT

A curable mixture and method of using the mixture are disclosed. In some embodiments, the mixture comprises a water soluble acrylate-based monomer, a water-soluble acrylamide-based monomer, or a mixture thereof, and has properties suitable for use as a tooth filling after curing.

### INCORPORATION BY REFERENCE TO ANY PRIORITY APPLICATIONS

Any and all applications for which a foreign or domestic priority claim is identified, for example, in the Application Data Sheet or Request as filed with the present application are hereby incorporated by reference under 37 CFR 1.57, and Rules 4.18 and 20.6. U.S. Provisional Application No. 63/109,794, filed Nov. 4, 2020, U.S. Provisional Application No. 63/109,797, filed Nov. 4, 2020, U.S. Provisional Application No. 63/109,802, filed Nov. 4, 2020, U.S. Provisional Application No. 63/115,493, filed Nov. 18, 2020, and U.S. Provisional Application No. 63/247,709, filed Sep. 23, 2021, are hereby incorporated by reference in their entirety.

### BACKGROUND

In conventional endodontic procedures, an opening is drilled through the crown of a diseased tooth, and endodontic files are inserted into the root canal system to open the canal spaces and remove organic material therein. The root canal is then filled with solid matter such as gutta percha and an obturation material, and the tooth is restored. However, this procedure may not remove all organic material from the canal spaces, which can lead to post-procedure complications such as infection. In addition, motion of the endodontic file may force organic material through an apical opening into periapical tissues. In some cases, the end of the endodontic file itself may pass through the apical opening. Such events may result in trauma to the soft tissue near the apical opening and lead to post-procedure complications.

Current treatment techniques for tooth decay (caries) generally include mechanical removal of the caries and diseased tissue (e.g., using dental burs, excavators, etc.), which will expose healthy dentin. However, the bur (or other mechanical instrument) may not differentiate between diseased and healthy dentin, and other instruments such as excavators and explorers may not be able to accurately determine the extent to which tooth removal should continue. This may result in either incomplete removal of caries or overly-aggressive removal of healthy dentin, which may in turn reduce the longevity of the tooth. The removed portions of the tooth can then be filled with solid matter such as composite, gold, porcelain, etc., and the tooth can be restored. However, this procedure may not remove all decayed material from the tooth, which combined with inadequate penetration of the restorative material can result in bacterial leakage and subsequently post-procedure complications such as infection or recurrent caries. In part to minimize the risk of reinfection, endodontic material placement typically requires the use of a gutta percha point to encourage penetration of the obturation material into lateral canals and isthmi. In addition, the use of a dental drill and anesthetics may be uncomfortable for the patient. Various filling spaces within or adjacent to a tooth can benefit from improvements in dental treatment techniques. Examples of such filling spaces include but are not limited to root canals, cavities resulting from the removal of caries, other openings such as cracks and gaps, and/or missing portions of teeth (e.g., resulting from fracture and/or wear). Accordingly, it can be advantageous to provide improved compositions, methods and apparatus for treating dental decay.

More recently, dental apparatuses have been developed that can deliver a curable mixture to a treatment region without the necessity of an obturation point. (See U.S. Pat. No. 9,877,801, the entire contents of which are hereby incorporated herein by reference for all purposes). Various formulations are known that can be used as curable mixtures. However, the compatibility of current materials with the new technology is less than desired. Thus, the need for more advanced obturation materials is needed.

### SUMMARY

Various non-limiting aspects of the present disclosure are provided to illustrate features of the disclosed compositions, apparatus and methods. Examples of compositions comprising curable materials for filling a tooth space are provided. A method of using the compositions for endodontic treatment, or for filling a dental treatment region comprising a tooth space such as a cavity, root canal, or crack, is provided. A method of using a dental apparatus to deliver the curable material to a tooth region is also provided. The dental apparatus may comprise a pressure wave generator to generate pressure waves to deliver the curable material throughout the tooth space. Further, a method for supplying a two-part curable composition to a dental apparatus to fill a tooth space with a curable mixture is provided.

In one aspect, a curable mixture of ingredients is described. The curable mixture includes: (a) a water-soluble acrylate-based monomer, a water-soluble acrylamide-based monomer, a water-soluble chelating monomer, or a mixture thereof; (b) a free-radical polymerization initiator; (c) a radiopaque material, wherein the radiopaque material is selected from the group consisting of 2,4,6-Triiodo-5-[(1-oxo-2-propen-1-yl)amino]-1,3-benzenedicarboxylic acid, 5-acryloylamino-N,N′-bis-[(2-hydroxy-1,1-bis-hydroxymethyl-ethylcarbamoyl)-methyl]-2,4,6-triiodoisophthalamide, 5-acryloylamino-N-(2,3-dihydroxypropyl)-N′-(2-hydroxy-1,1-bis-hydroxymethylethyl)-2,4,6-triiodoisophthalamide, and combinations thereof; and (d) an aqueous carrier; wherein the ingredients (a), (b), (c), and (d) are selected to provide the curable mixture with properties suitable for use as a tooth filling after curing the mixture to form a cured mixture by polymerization of ingredient (a) that is initiated by ingredient (b).

In another aspect, an obturation material for use as a radiopaque tooth filling after curing is described. The obturation material, provided as two or more liquid parts, includes: (a) a water-soluble acrylate-based monomer, a water-soluble acrylamide-based monomer, a water-soluble chelating monomer, or a mixture thereof, selected from the group consisting of a multi-arm poly(ethylene glycol) acrylate, a multi-arm poly(ethylene glycol) acrylamide, an acrylate functionalized polyether, an acrylamide functionalized polyether, a methacrylamide functionalized polyether, and combinations thereof; (b) a free-radical polymerization initiator; (c) a radiopaque material; and (d) an aqueous carrier; wherein at least one liquid part has a viscosity less than 100 cP (at 25° C.), wherein (a), (b), (c), and (d) are selected to form a curable mixture, and wherein the obturation material is curable in a tooth by polymerization of ingredient (a) that is initiated by ingredient (b).

In another aspect, a curable mixture of ingredients is described. The curable mixture includes: (a) 14 wt. % to 65 wt. % poly(ethylene glycol) diacrylate; (b) 15 wt. % to 45 wt. % of 5-acrylamido-2,4,6-triiodo isophthalic acid; (c) 0.2 wt. % to 6 wt. % a free-radical polymerization initiator; and (d) 20 wt. % to 60 wt. % of an aqueous carrier, wherein the ingredients are selected to provide the curable mixture with properties suitable for use as a tooth filling material after curing.

In another aspect, a curable mixture of ingredients is described. The curable mixture includes: (a) 14 wt. % to 65 wt. % poly(ethylene glycol) diacrylate; (b) 0.2 wt. % to 6 wt. % potassium persulfate; (c) 0.3 wt. % to 1.0 wt. % 1-phenyl-1,2-propanedione (PPD); (d) 15 wt. % to 50 wt. % of 5-acrylamido-2,4,6-triiodo isophthalic acid; and (e) 20 wt. % to 60 wt. % of an aqueous carrier, wherein the ingredients are selected to provide properties suitable for use as a tooth filling material after curing.

In another aspect, an obturation material for use as a radiopaque tooth filling after curing is described. The obturation material, provided as two or more liquid parts, includes: (a) 15 wt. % to 65 wt. % poly(ethylene glycol) diacrylate; (b) 15 wt. % to 45 wt. % of 5-acrylamido-2,4,6-triiodo isophthalic acid; (c) 0.2 wt. % to 6 wt. % a first free-radical polymerization initiator; (d) 0.3 wt. % to 1.0 wt. % a second free-radical polymerization initiator that is a light initiator; and (e) 20 wt. % to 60 wt. % of an aqueous carrier, wherein at least one liquid part comprising (a) through (d) has a viscosity less than 60 cP (at 25° C.); wherein (a), (b), (c), and (d) are selected to form a curable mixture; and wherein the obturation material is curable in a tooth by a polymerization of ingredient (a) that is initiated by ingredients (c) and (d).

In another aspect, a curable mixture of ingredients is described. The curable mixture includes: (a) 20 wt. % to 65 wt % 3-arm poly(ethylene glycol) acrylate; (b) 15 wt % to 30 wt. % of 5-acrylamido-2,4,6-triiodo isophthalic acid; (c) at least one free-radical polymerization initiator in an amount of 0.2 wt. % to 1.5 wt %; and (d) 20 wt % to 60 wt % of an aqueous carrier.

In another aspect, an obturation material for use as a radiopaque tooth filling after curing is described. The obturation material, provided as two or more liquid parts, includes: (a) 20 wt. % to 65 wt % 3-arm poly(ethylene glycol) acrylate; (b) 15 wt % to 30 wt. % of 5-acrylamido-2,4,6-triiodo isophthalic acid; (c) 0.2 wt. % to 1.5 wt % of a first free-radical polymerization initiator; (d) optionally, 0.3 wt % to 1.0 wt % of a second free-radical polymerization initiator that is a light initiator; (e) optionally, 0.2 wt % to 6% of a co-initiator; and (f) 20 wt % to 60 wt % of an aqueous carrier, wherein at least one liquid part has a viscosity less than 60 cP (at 25° C.), wherein (a), (b), (c), (d) and (e) are selected to form a curable mixture, and wherein the obturation material is curable in a tooth by a polymerization of ingredient (a) that is initiated by ingredient (c) and/or (d).

In another aspect, a curable mixture of ingredients is described. The curable mixture includes: (a) 15 wt. % to 65 wt. % water soluble acrylamide-based monomer; (b) 15 wt. % to 45 wt. % water soluble ionic or non-ionic iodinated X-ray contrast acrylate monomer; (c) 0.2 wt. % to 6 wt. % a free-radical polymerization initiator; and (d) 15 wt. % to 60 wt. % of an aqueous carrier, wherein the ingredients are selected to provide the curable mixture with properties suitable for use as a tooth filling after curing of the curable mixture to form a cured mixture.

In another aspect, an obturation material for use as a radiopaque tooth filling after curing is described. The obturation material, provided as two or more liquid parts, includes: (a) 15 wt. % to 65 wt. % water soluble acrylamide-based monomer; (b) 15 wt % to 30 wt. % of 5-acrylamido-2,4,6-triiodo isophthalic acid; (c) 0.2 wt. % to 1.5 wt % of a free-radical polymerization initiator; (d) optionally, 0.3 wt % to 1.0 wt % of a second free-radical polymerization initiator that is a light initiator; (e) optionally, 0.2 wt % to 6% of a co-initiator; and (f) 20 wt % to 60 wt % of an aqueous carrier, wherein at least one liquid part has a viscosity less than 60 cP (at 25° C.), wherein (a), (b), (c), (d) and (e) are selected to form a curable mixture, and wherein the obturation material is curable in a tooth by a polymerization of ingredient (a) that is initiated by ingredient (c) and/or (d).

In another aspect, a curable mixture of ingredients is described. The curable mixture includes: (a) 15 wt. % to 65 wt. % water soluble acrylate-based monomer, acrylamide-based monomer, or a combination thereof; (b) 15 wt. % to 45 wt. % water soluble iodinated X-ray contrast acrylate monomer; (c) 0.2 wt. % to 6 wt. % a free-radical polymerization initiator; and (d) 15 wt. % to 60 wt. % of an aqueous carrier, wherein the ingredients are selected to provide properties suitable for use as a tooth filling material after curing.

In another aspect, an obturation material, for use as a tooth filling material after curing is described. The obturation material includes a curable mixture provided as two or more liquid parts, including: (a) a water-soluble acrylate-based monomer, a water-soluble acrylamide-based monomer, a water-soluble chelating monomer, or a mixture thereof; (b) calcium silicate dispersed in a non-aqueous carrier; (c) a free-radical polymerization initiator; (d) a water-soluble radiopaque material; and (e) a low viscosity liquid; wherein components (a) and (b) are selected to provide properties suitable for use as a tooth filling material after curing to form a cured mixture by polymerization of ingredient (a) that is initiated by ingredient (c).

In another aspect, a curable mixture of ingredients is described. The curable mixture includes: (a) a water-soluble acrylate-based monomer, a water-soluble acrylamide-based monomer, a water-soluble chelating monomer, or a mixture thereof; (b) a free-radical polymerization initiator; (c) a radiopaque material; and (d) an aqueous carrier; wherein the ingredients (a), (b), (c), and (d) are selected to provide the curable mixture with properties suitable for use as a tooth filling after curing the mixture to form a cured mixture by polymerization of ingredient (a) that is initiated by ingredient (b); and wherein the radiopaque material comprises at least 3 wt % of the curable mixture.

Those skilled in the art will recognize that embodiments disclosed herein may achieve one advantage or group of advantages as taught herein without necessarily achieving other advantages as may be taught or suggested herein. Further, the foregoing is intended to summarize certain disclosed embodiments and is not intended to limit the scope of the embodiments, which may be disclosed in greater detail below.

### BRIEF DESCRIPTION OF THE DRAWINGS

The foregoing and other features, aspects, and advantages of embodiments of the apparatus, compositions and methods of filling spaces in teeth are described in detail below with reference to the drawings, which are intended to illustrate and not to limit the embodiments. The drawings comprise the following FIGS. in which:

FIG. 1A is a schematic diagram of a dental treatment system for treating a root canal, according to various embodiments disclosed herein.

FIG. 1B is a schematic diagram of a system that includes components configured to clean unhealthy or undesirable material from a treatment region on an exterior surface of the tooth.

FIG. 1C is a schematic diagram of the system of FIG. 1B, in which the system is configured to fill a treated carious region of the tooth.

FIG. 2A is a schematic top plan view of a delivery device that can be used to combine a first composition with a second composition to form a curable mixture and to fill a treatment region.

FIG. 2B is a schematic side sectional view of a portion of the delivery device of FIG. 2A.

Throughout the drawings, reference numbers may be reused to indicate a general correspondence between referenced elements. The drawings are provided to illustrate examples of embodiments described herein and are not intended to limit the scope of the disclosure.

### DETAILED DESCRIPTION

To protect the long-term health of the tooth, it can be advantageous to substantially fill the filling space or spaces of a tooth created from removal of caries, through root canal treatment, and/or natural wear. When the restoration follows a root canal treatment it can be important to fill not only the major canal spaces, but also any minor cracks and open spaces in the tooth with a filling material. Similarly, when the restoration follows a caries treatment it can be important to fill the resulting dental spaces in order to provide dimensional stability and/or structural integrity to the tooth.

In various embodiments, the filling material is an obturation material. The term “obturation material” refers to a material that is configured to fill root canals, restore carious lesions, and/or modify the surface of the tooth. The obturation material may be a polymerizable restorative composition that includes a curable mixture that is cured or hardened to form the final material, which may be referred to as a cured mixture, mixture or “tooth filling.” It should be appreciated that terms such as setting, curing, hardening, cross-linking, polymerizing, and the like, refer to processes by which the obturation material components are transformed into a final hardened mixture in the tooth. In this context, an obturation material that is “suitable for use as a tooth filling” comprises a corresponding cured or hardened state having properties that meet standards set by an appropriate regulatory body (e.g., ISO 6876:2012—Dental root canal sealing materials). A cured obturation material having such properties is considered to meet the standards regardless of whether the regulatory body has provided official notification to that effect.

In some embodiments, various obturation material compositions or components thereof as described herein can be formed into a coherent collimated jet for delivery to a tooth space. For example, in an embodiment, an obturation material composition or components thereof, as described herein, can be formed into a liquid jet that forms a substantially parallel beam (e.g., is “collimated”) over distances ranging from about 0.01 cm to about 10 cm. In some embodiments, the velocity profile transverse to the propagation axis of the jet is substantially constant (e.g., is “coherent”). For example, in some implementations, away from narrow boundary layers near the outer surface of the jet (if any), the jet velocity is substantially constant across the width of the jet. Therefore, in certain advantageous embodiments, the liquid jet (e.g., as delivered by an apparatus as described herein) may comprise a coherent, collimated jet (a “CC jet”). In some implementations, the CC jet may have velocities in a range from about 100 meters per second (m/s) to about 300 m/s, for example, about 190 m/s in some embodiments. In some implementations, the CC jet can have a diameter in a range from about 5 microns to about 1000 microns, in a range from about 10 microns to about 100 microns, in a range from about 100 microns to about 500 microns, or in a range from about 500 microns to about 1000 microns. Further details with respect to CC jets that can be comprised of obturation material compositions or components thereof as described herein can be found in U.S. Patent Publication No. 2007/0248932, which is hereby incorporated by reference herein in its entirety for all that it discloses or teaches.

In some embodiments, an obturation material comprises two or more components that react with one another to form a hardened obturation material. In other embodiments, the obturation or filling material may comprise a composition that is curable from a flowable state to a hardened state by exposure to an energy source such as light or heat, or both light and heat. In one embodiment, a curable mixture of ingredients comprises: (a) a water soluble acrylate-based monomer, a water-soluble acrylamide-based monomer, a water-soluble chelating monomer, or a mixture thereof; (b) free-radical polymerization initiator; (c) a radiopaque material; and (d) an aqueous carrier, wherein the ingredients (a), (b), (c), and (d) are selected to provide the curable mixture with properties suitable for use as a tooth filling after curing by polymerization of ingredient (a) that is initiated by ingredient (b). In one aspect, the cured obturation material may comprise a hydrogel material. The hydrogel material may comprise a hydrophilic polymer matrix or macromolecule that holds a large amount of water while maintaining structure as a hard gel, which is biocompatible and/or resistant to degradation in vivo.

Various water-soluble acrylate-based monomers and mixtures thereof are suitable for use in forming the curable mixture of ingredients. In some embodiments, the water-soluble acrylate-based monomer is a diacrylate monomer or a triacrylate monomer. In some embodiments, the water-soluble acrylate-based monomer is an acrylate monomer that is cationically charged, for example, an acrylate monomer that contains a quaternary ammonium group and a counterion. Examples include, but are not limited to, [2-(acryloyloxy)ethyl] trimethylammonium halide (e.g., halide is chloride counterion), [2-(methacryloyloxy)ethyl] trimethylammonium halide (e.g., halide is chloride counterion), [2-(acryloyloxy)ethyl] trimethylammonium methyl sulfate, and [2-(methacryloyloxy)ethyl] trimethylammonium methyl sulfate, and a combination of one or more thereof. In various embodiments, the cationically charged water-soluble acrylate-based monomer is present in an amount effective to inhibit bacterial growth in the resulting cured mixture. In some embodiments, the water-soluble acrylate-based monomer is an acrylate monomer that is uncharged. In some embodiments, the water-soluble acrylate-based monomer may be a diacrylate or triacrylate, including poly(ethylene glycol) diacrylate (PEGDA), poly(ethylene glycol) dimethacrylate, (hydroxyethyl)methacrylate (HEMA), multi-arm poly(ethylene glycol) acrylate, including 3-arm PEG acrylate and 3-arm PEG methacrylate, or a mixture thereof. 3-arm PEG (meth)acrylate may have one, two or three functionalized arms (e.g. ethoxylated trimethylolpropane triacrylate (ETT)), and the core may comprise glycerol or trimethylolpropane. In some embodiments, the water-soluble acrylate-based monomer is a high-molecular weight poly(ethylene glycol) diacrylate (e.g., having a Mn of 700, or greater than 700). In some embodiments, the water soluble acrylate-based monomer, such as a triacrylate or diacrylate, may be present in an amount from 1 wt % to 75 wt %, 1 wt % to 60 wt %, 1 wt % to 50 wt %, 1 wt % to 25 wt %, 15 wt % to 75 wt %, 15 wt % to 60 wt %, 15 wt % to 58 wt %, 20 wt % to 60 wt %, 20 wt % to 58 wt %, 20 wt % to wt %, 12 wt % to 27 wt %, 12 wt % to 24 wt %, 12 wt % to 21 wt %, 15 wt % to 27 wt %, 15 wt % to 24 wt %, 15 wt % to 21 wt %, 20 wt % to 45 wt %, 20 wt % to 40 wt %, 20 wt % to 35 wt %, 25 wt % to 45 wt %, 25 wt % to 40 wt %, 25 wt % to 35 wt %, 0.2 wt % to 10 wt %, or from or 0.2 wt % to 5 wt %, based on the total weight of the curable mixture or a total weight of one part of the curable mixture. In some embodiments, the water soluble acrylate-based monomer, such as a triacrylate or diacrylate, may be present in an amount of, or of about, 0.1 wt %, 0.2 wt %, 0.5 wt %, 1 wt %, 5 wt %, 10 wt %, 12 wt %, 15 wt %, 20 wt %, 21 wt %, 24 wt %, 25 wt %, 27 wt %, 30 wt %, 35 wt %, 40 wt %, 45 wt %, 50 wt %, 55 wt %, 58 wt %, 60 wt %, 65 wt %, 70 wt %, 75 wt %, wt %, 85 wt %, 90 wt %, 95 wt % or 100 wt. %, based on the total weight of the curable mixture or a total weight of one part of the curable mixture, or any range of values therebetween.

Various water-soluble acrylamide-based monomers, and mixtures thereof, are suitable for use in the curable mixture of ingredients. In some embodiments, the water-soluble acrylamide-based monomer is an acrylamide monomer that is cationically charged, and for example, may contain a quaternary ammonium group and a counterion. Examples include, but are not limited to, 3-acrylamidopropyl trimethylammonium halide (e.g., halide is chloride counterion), 3-methacrylamidopropyl trimethylammonium halide (e.g., halide is chloride counterion), 3-acrylamidopropyl trimethylammonium methyl sulfate, and 3-methacrylamidopropyl trimethylammonium methyl sulfate, and a combination of one or more thereof. In some embodiments, a cationically charged water-soluble acrylamide-based monomer is present in an amount effective to inhibit bacterial growth in the resulting cured mixture. The water-soluble acrylamide-based monomer may comprise 3-acrylamidopropyl trimethylammonium chloride, 3-methacrylamidopropyl trimethylammonium chloride, 3-acrylamidopropyl trimethylammonium methyl sulfate, 3-methacrylamidopropyl trimethylammonium methyl sulfate, or a combination thereof. In some embodiments, a water soluble acrylamide-based monomer may be present in an amount from 1 wt % to 60 wt %, 1 wt % to 20 wt %, 1 wt % to 15 wt %, 20 wt % to 58 wt %, 20 wt % to 50 wt %, 12 wt % to 27 wt %, 12 wt % to 24 wt %, 12 wt % to 21 wt %, 15 wt % to 27 wt %, 15 wt % to 24 wt %, 15 wt % to 21 wt %, 20 wt % to 45 wt %, 20 wt % to 40 wt %, 20 wt % to 35 wt %, 25 wt % to 45 wt %, 25 wt % to 40 wt %, wt % to 35 wt %, 0.2 wt % to 10 wt %, or from 0.2 wt % to 5 wt %, based on the total weight of the curable mixture or a total weight of one part of the curable mixture. In some embodiments, the water soluble acrylamide-based monomer may be present in an amount of, or of about, 0.1 wt %, 0.2 wt %, 0.5 wt %, 1 wt %, 5 wt %, 10 wt %, 12 wt %, 15 wt %, 20 wt %, 21 wt %, 24 wt %, 25 wt %, 27 wt %, 30 wt %, 35 wt %, 40 wt %, 45 wt %, 50 wt %, 55 wt %, 58 wt %, 60 wt %, 65 wt %, wt %, 75 wt %, 80 wt %, 85 wt %, 90 wt %, 95 wt % or 100 wt. %, based on the total weight of the curable mixture or a total weight of one part of the curable mixture, or any range of values therebetween.

Water soluble acrylamide-based monomers may include, but are not limited to, acrylamide, diacrylamide, triacrylamide, methacrylamide, poly(ethylene glycol)-acrylamide, poly(ethylene glycol) diacrylamide, poly(ethylene glycol) triacrylamide, multi-arm poly(ethylene glycol) acrylamide-based monomers having one or more functional arms, such as 3-arm poly(ethylene glycol) acrylamide, 3-arm poly(ethylene glycol) triacrylamide, and 4-arm poly(ethylene glycol) acrylamide.

In some embodiments, the monomer (e.g. water-soluble acrylate-based monomer, the water-soluble acrylamide-based monomer, and the water-soluble chelating monomer) are selected from a multi-arm poly(ethylene glycol) acrylate, a multi-arm poly(ethylene glycol) acrylamide, an acrylate functionalized polyether, an acrylamide functionalized polyether, and a methacrylamide functionalized polyether. In some embodiments, the multi-arm poly(ethylene glycol) acrylate is selected from 3-arm PEG acrylate and 3-arm PEG methacrylate. In some embodiments, the multi-arm poly(ethylene glycol) acrylamide is selected from 3-arm poly(ethylene glycol) acrylamide, 3-arm poly(ethylene glycol) triacrylamide, and 4-arm poly(ethylene glycol) acrylamide.

Further examples of acrylamide-based monomers include but are not limited to N,N′-(dimethyl)-ethylenebisacrylamide, bis[2-(2-methyl-acrylamino)-ethoxycarbonyl]-hexamethylenediamine, and N,N′-diethyl-1,3-propylene-bis-acrylamide. (Meth)acrylamide compounds may be represented by the general formulas I and II:

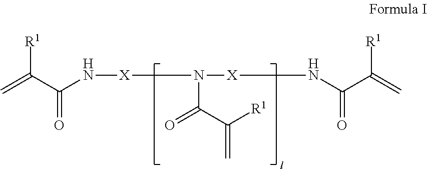

wherein R 1<represents a hydrogen atom or a methyl group, 1 represents an integer of 1 to 6, X represents an optionally substituted, linear or branched C 1 to C 8 alkylene group, the plurality of R 1< may be the same or different, and the plurality of X may be the same or different,

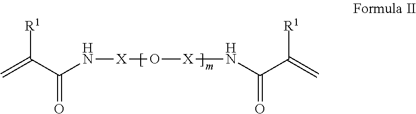

wherein m represents 2 or 3, R 1< and X are as defined above, the plurality of R 1< may be the same or different, and the plurality of X may be the same or different. An acrylamide-methacrylic acid ester compound may be represented by Formula III

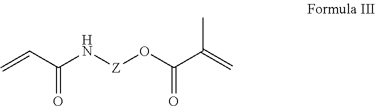

wherein Z is an optionally substituted, linear or branched C1 to C 8 aliphatic group or an optionally substituted aromatic group, the aliphatic group being optionally interrupted by at least one linking group selected from the group consisting of —O—, —S—, —CO—, —OO—O—, —O—CO—, —NR 2<-, —CO—NR 2<-, —NR 2<—CO—, —CO—O—NR 2<-, —O—CONR 2<-, and —NR 2<—CO—NR 2<-, and R 2<represents a hydrogen atom, or an optionally substituted, linear or branched C1 to C8 aliphatic group. X is a moiety which may be selected for adjusting the hydrophilicity of the (meth)acrylamide compound (a). In some embodiments X in formula (1) is an optionally substituted, linear or branched C1 to C5 alkylene group, such as an optionally substituted, linear or branched C2 to C4 alkylene group, or an unsubstituted linear C2 to C4 alkylene group.

Examples of the linear or branched C1 to C8 alkylene group include methylene, methylmethylene, ethylene, 1-methylethylene, 2-methylethylene, trimethylene, 1-ethylethylene, 2-ethylethylene, 1,2-dimethylethylene, 2,2-dimethylethylene, 1-methyltrimethylene, 2-methyltrimethylene, 3-methyltrimethylene, tetramethylene, 1-propylethylene, 2-propylethylene, 1-ethyl-1-methylethylene, 1-ethyl-2-methylethylene, 1,1,2-trimethylethylene, 1,2,2-trimethylethylene, 1-ethyltrimethylene, 2-ethyltrimethylene, 3-ethyltrimethylene, 1,1-dimethyltrimethylene, 1,2-dimethyltrimethylene, 1,3-dimethyltrimethylene, 2,3-dimethyltrimethylene, 3,3-dimethyltrimethylene, 1-methyltetramethylene, 2-methyltetramethylene, 3-methyltetramethylene, 4-methyltetramethylene, pentamethylene, 1-butylethylene, 2-butylethylene, 1-methyl-1-propylethylene, 1-methyl-2-propylethylene, 2-methyl-2-propylethylene, 1,1-diethylethylene, 1,2-diethylethylene, 2,2-diethylethylene, 1-ethyl-1,2-dimethylethylene, 1-ethyl-2,2-dimethylethylene, 2-ethyl-1,1-dimethylethylene, 2-ethyl-1,2-dimethylethylene, 1, 1,2,2-tetramethylethylene, 1-propyltrimethylene, 2-propyltrimethylene, 3-propyltrimethylene, 1-ethyl-1-methyltrimethylene, 1-ethyl-2-methyltrimethylene, 1-ethyl-3-methyltrimethylene, 2-ethyl-1-methyltrimethylene, 2-ethyl-2-methyltrimethylene, 2-ethyl-3-methyltrimethylene, 3-ethyl-1-methyltrimethylene, 3-ethyl-2-methyltrimethylene, 3-ethyl-3-methyltrimethylene, 1, 1,2-trimethyltrimethylene, 1,1,3-trimethyltrimethylene, 1,2,2-trimethyltrimethylene, 1,2,3-trimethyltrimethylene, 1,3,3-trimethyltrimethylene, 2,2,3-trimethyltrimethylene, 2,3,3-trimethyltrimethylene, 1-ethyltetramethylene, 2-ethyltetramethylene, 3-ethyltetramethylene, 4-ethyltetramethylene, 1,1-dimethyltetramethylene, 1,2-dimethyltetramethylene, 1,3-dimethyltetramethylene, 1,4-dimethyltetramethylene, 2,2-dimethyltetramethylene, 2,3-dimethyltetramethylene, 2,4-dimethyltetramethylene, 3,3-dimethyltetramethylene, 3,4-dimethyltetramethylene, 4,4-dimethyltetramethylene, 1-methylpentamethylene, 2-methylpentamethylene, 3-methylpentamethylene, 4-methylpentamethylene, 5-methylpentamethylene, hexamethylene, 2,2,3-trimethyltetramethylene, 3-ethylpentamethylene, 2,2-dimethylpentamethylene, 2,3-dimethylpentamethylene, 2,4dimethylpentamethylene, 3,3-dimethylpentamethylene, 2-methylhexamethylene, 3-methylhexamethylene, heptamethylene, 2,2,3,3-tetramethyltetramethylene, 2,2,3-trimethylpentamethylene, 2,2,4-trimethylpentamethylene, 2,3,3-trimethylpentamethylene, 2,3,4-trimethylpentamethylene, 3-ethyl-2-methylpentamethylene, 3-ethyl-3-methylpentamethylene, 2,2-dimethylhexamethylene, 2,3-dimethylhexamethylene, 2,4-dimethylhexamethylene, 2,5-dimethylhexamethylene, 3,3-dimethylhexamethylene, 3,4-dimethylhexamethylene, 3-ethylhexamethylene, 2-methylheptamethylene, 3-methylheptamethylene, 4-methylheptamethylene, and octamethylene groups. In some embodiments, acrylamides include methylene, methylmethylene, ethylene, 1-methylethylene, 2-methylethylene, trimethylene, 1-ethylethylene, 2-ethylethylene, 1,2-dimethylethylene, 2,2-dimethylethylene, 1-methyltrimethylene, 2-methyltrimethylene, 3-methyltrimethylene, and tetramethylene groups.

Water-soluble polyether having a functionable end group such as a primary amine (—NH2) may be functionalized to form acrylamide or methacrylamide. O,O′-bis(2-aminopropyl) polypropylene glycol-block-polyethylene glycol-block-polypropylene glycol of Formula (IV),

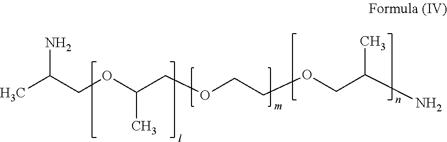

may be converted to an acrylamide by reacting with acryloyl chloride in solution, and in one embodiment has a core (polyethylene glycol), where m is from about 15 to 45 units, such as 39 units, and 1+n (polypropylene glycol) is approximately 6. O,O′-bis(2-aminopropyl) polypropylene glycol-block-polyethylene glycol-block-polypropylene glycol (Mn=800), liquid at ambient temperature, may be commercially available under the trade name Jeffamine® ED-900 (trademark Huntsman; Sigma-Aldrich; Milwaukee, WI; product #14527). O,O′-bis(2-aminopropyl) polypropylene glycol-block-polyethylene glycol-block-polypropylene glycol (Mn=1900), solid at ambient temperate, may be commercially available under the tradename Jeffamine® ED 2003 (trademark Huntsman; Sigma-Aldrich Milwaukee, WI; product #14529).

In some embodiments, the acrylate functionalized polyether is selected from a Jeffamine® ED-900 acrylate and a Jeffamine® ED 2003 acrylate. In some embodiments, the acrylamide functionalized polyether is selected from a Jeffamine® ED-900 acrylamide and a Jeffamine® ED 2003 acrylamide. In some embodiments, the methacrylamide functionalized polyether is selected from a Jeffamine® ED-900 methacrylamide and a Jeffamine® ED 2003 methacrylamide. In some embodiments, the acrylate functionalized polyether is selected from Jeffamine® ED-900 diacrylate and Jeffamine® ED 2003 diacrylate. In some embodiments, the acrylamide functionalized polyether is selected from Jeffamine® ED-900 triacrylamide, and Jeffamine® ED 2003 triacrylamide.

Methods for converting O,O′-bis(2-aminopropyl) polypropylene glycol-block-polyethylene glycol-block-polypropylene glycol into acrylamides, are described, for example, in Elbert D. L and Hubbell J. A, “Conjugate Addition Reactions Combined with Free-Radical Cross-Linking for the Design of Materials for Tissue Engineering”, Biomacromolecules 2001, 2, 430-441. Water soluble diacrylamide may be synthesized, for example, from linear PEG 1000 having hydroxy end groups (Sigma-Aldrich, #8.07488) and converting it into di-mesylate, then diamine, and then into diacrylamide. Methods are provided, for example, in Elbert D. L and Hubbell J. A, “Conjugate Addition Reactions Combined with Free-Radical Cross-Linking for the Design of Materials for Tissue Engineering”, Biomacromolecules 2001, 2, 430-441. In a further embodiment, a poly(ethylene glycol) triacrylamide suitable for used in the obturation materials, may be made from polyethylene glycol trimethylolpropane triether or trimethylolpropane ethoxylate (20/3 EO/OH) (average Mn ˜1,014), that is first converted into PEG tri-mesylate, then PEG tri-amine, and finally, PEG triacrylamide. Further methods for synthesizing water soluble acrylate monomers include, but are not limited to methods provided in U.S. Pat. No. 8,470,035 issued Jun. 25, 2013; Liang, N, Flynn L. E. and Gillies E. R., “Neutral, water-soluble poly(ester amide) hydrogels for cell encapsulation”, European Polym. J, 136 (2020) 109899, using toluenesulfonyl chloride for converting hydroxy group into amine group.

Various water-soluble chelating monomers and mixtures thereof are suitable for use in the curable mixture of ingredients. Examples of a chelating monomer include but are not limited to 4-methacryloxyethyl trimellitic acid (4-MET) and glycerol phosphate dimethacrylate (GPDM). In various embodiments, the chelating monomer is used in an amount effective to enhance adhesion of the resulting curable mixture to a surface of a tooth. In some embodiments, the water-soluble chelating monomer may be present in an amount from 1 wt % to wt %, 1 wt % to 60 wt %, 1 wt % to 50 wt %, 1 wt % to 25 wt %, 15 wt % to 75 wt %, 15 wt % to wt %, 15 wt % to 58 wt %, 20 wt % to 60 wt %, 20 wt % to 58 wt %, 20 wt % to 50 wt %, 12 wt % to 27 wt %, 12 wt % to 24 wt %, 12 wt % to 21 wt %, 15 wt % to 27 wt %, 15 wt % to 24 wt %, 15 wt % to 21 wt %, 20 wt % to 45 wt %, 20 wt % to 40 wt %, 20 wt % to 35 wt %, 25 wt % to 45 wt %, wt % to 40 wt %, 25 wt % to 35 wt %, 0.2 wt % to 10 wt %, or from or 0.2 wt % to 5 wt %, based on the total weight of the curable mixture or a total weight of one part of the curable mixture. In some embodiments, the water-soluble chelating monomer may be present in an amount of, or of about, 0.1 wt %, 0.2 wt %, 0.5 wt %, 1 wt %, 5 wt %, 10 wt %, 12 wt %, 15 wt %, 20 wt %, 21 wt %, 24 wt %, 25 wt %, 27 wt %, 30 wt %, 35 wt %, 40 wt %, 45 wt %, 50 wt %, 55 wt %, 58 wt %, 60 wt %, 65 wt %, 70 wt %, 75 wt %, 80 wt %, 85 wt %, 90 wt %, 95 wt % or 100 wt. %, based on the total weight of the curable mixture or a total weight of one part of the curable mixture, or any range of values therebetween.

In one embodiment of an obturation material, component (a) comprises a water-soluble monomer mixture that comprises two or more of a water-soluble acrylate-based monomer, a water-soluble acrylamide-based monomer, and a water-soluble chelating monomer. For example, component (a) may comprise a water-soluble monomer mixture that comprises a water-soluble acrylate monomer and a water-soluble acrylamide-based monomer. In another embodiment of an obturation material, component (a) comprises a water-soluble monomer mixture that comprises two water soluble acrylate monomers, such as a water-soluble diacrylate monomer and a water-soluble triacrylate monomer.

Various radiopaque materials and mixtures thereof are suitable for use in the curable mixture. In some embodiments, the radiopaque material may be a radiopaque monomer or a radiopaque salt. In some embodiments, the radiopaque material may be water-soluble, such as a water-soluble radiopaque monomer or radiopaque salt. The radiopaque material may be water-insoluble, such as a water-insoluble radiopaque monomer or radiopaque salt (e.g., barium sulfate). The radiopaque salt may be a radio-dense iodide or barium salt, such as calcium iodide, potassium iodide, sodium iodide, barium sulfate or barium chloride. In other embodiments, radiopaque salts may include (MRI) radio-contrast agents such as a gadolinium salt, and/or a diatrizoate sodium type agent (such as sodium diatrizoate hydrate). Other radiopaque materials include, but are not limited to, radiopaque aromatic acids, such as a water soluble radiopaque aromatic acid derived of (meth)acrylate, iodinated benzene derivative, and tri iodinated compounds, including 2,4,6-Triiodo-5-[(1-oxo-2-propen-1-yl)amino]-1,3-benzenedicarboxylic acid (i.e. CAS no. 783279-02-7 or 5-amino-2,4,6-triiodoisophthalic acid), and an ionic iodinated x-ray contrast acrylate monomer, such as 5-acrylamido-2,4,6-triiodo isophthalic acid, for example as disclosed in European Patent Application EP 0 436 316 A1, hereby incorporated in its entirety herein. Tri iodinated compounds may also include an ionic iodinated x-ray contrast acrylate monomer, such as 5-acryloylamino-N,N′-bis-[(2-hydroxy-1,1-bis-hydroxymethyl-ethylcarbamoyl)-methyl]-2,4,6-triiodoisophthalamide and 5-acryloylamino-N-(2,3-dihydroxypropyl)-N′-(2-hydroxy-1,1-bis-hydroxymethylethyl)-2,4,6-triiodoisophthalamide. In some embodiments, the radiopaque material may be selected from 2,4,6-Triiodo-54(1-oxo-2-propen-1-yl)amino]-1,3-benzenedicarboxylic acid, 5-acryloylamino-N,N′-bis-[(2-hydroxy-1,1-bis-hydroxymethyl-ethylcarbamoyl)-methyl]-2,4,6-triiodoisophthalamide, 5-acryloylamino-N-(2,3-dihydroxypropyl)-N′-(2-hydroxy-1,1-bis-hydroxymethylethyl)-2,4,6-triiodoisophthalamide, and combinations thereof.

In one embodiment, 5-acryloylamino-N,N′-bis-[(2-hydroxy-1,1-bis-hydroxymethyl-ethylcarbamoyl)-methyl]-2,4,6-triiodoisophthalamide may be synthesized substantially according to the process below.

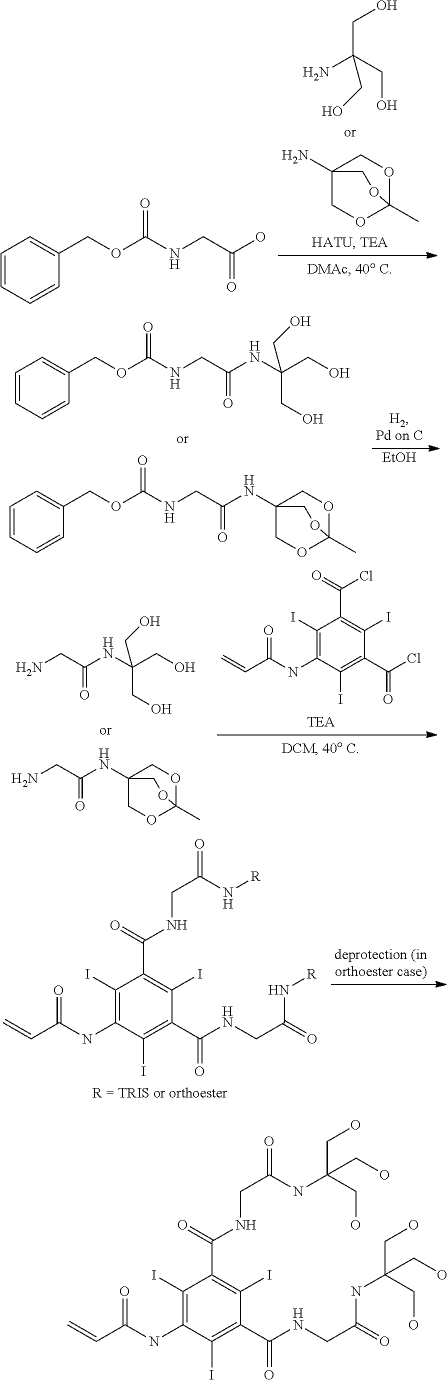

In another embodiment, 5-acryloylamino-N-(2,3-dihydroxypropyl)-N′-(2-hydroxy-1,1-bis-hydroxymethylethyl)-2,4,6-triiodoisophthalamide may be synthesized substantially according to the process below.

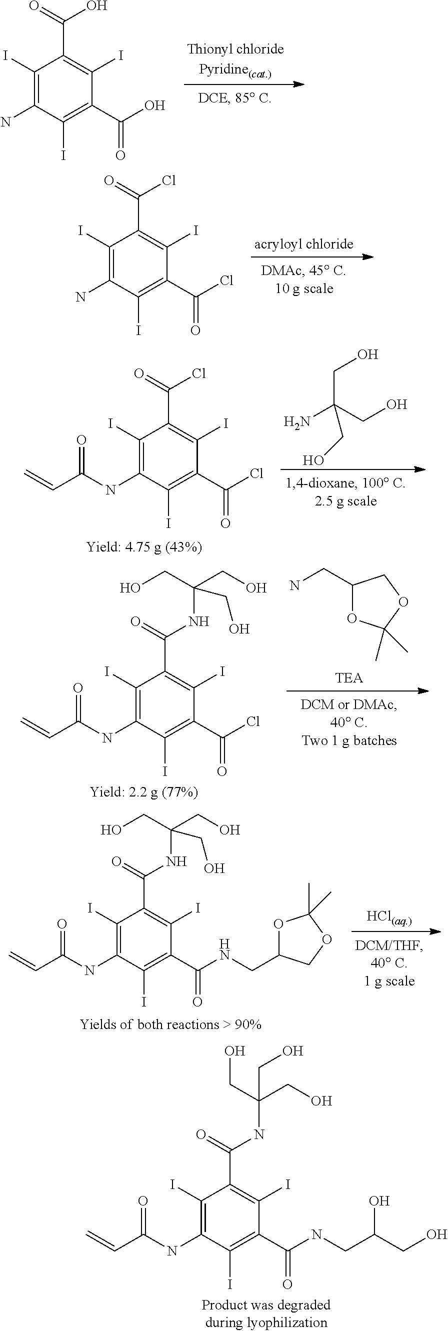

The radiopaque material may be included in the curable mixture in an amount effective to render the resulting cured mixture radiopaque, e.g., suitable for imaging by dental X-ray. The radiopacity of cured polymer materials described herein may be measured by ISO6876:2012, and in some embodiments, have a radiopacity greater than 1 mmAl, or greater than 2 mmAl, or greater than 3 mmAl. In some embodiments, a radiopaque material may be present in an amount from 1 wt % to 60 wt %, 1 wt % to 20 wt %, 1 wt % to 15 wt %, 20 wt % to 58 wt %, 20 wt % to 50 wt %, 12 wt % to 27 wt %, 12 wt % to 24 wt %, 12 wt % to 21 wt %, 15 wt % to 27 wt %, 15 wt % to 24 wt %, 15 wt % to 21 wt %, 20 wt % to 45 wt %, 20 wt % to 40 wt %, 20 wt % to 35 wt %, 25 wt % to 45 wt %, 25 wt % to 40 wt %, 35 wt % to 40 wt %, 35 wt % to 55 wt %, 25 wt % to 35 wt %, 0.2 wt % to 10 wt %, or from 0.2 wt % to 5 wt %, based on the total weight of the curable mixture or a total weight of one part of the curable mixture. In some embodiments, the radiopaque material may be present in an amount of, or of about, 0.1 wt %, 0.2 wt %, 0.5 wt %, 1 wt %, 5 wt %, 10 wt %, 12 wt %, 15 wt %, 20 wt %, 21 wt %, 24 wt %, 25 wt %, 27 wt %, 30 wt %, 35 wt %, 40 wt %, 45 wt %, 50 wt %, 55 wt %, 58 wt %, 60 wt %, 65 wt %, 70 wt %, 75 wt %, 80 wt %, wt %, 90 wt % or 100 wt. %, based on the total weight of the curable mixture or a total weight of one part of the curable mixture, or any range of values therebetween. In some embodiments, the radiopaque material also acts as a nanofiller material. In some embodiments, the radiopaque material is soluble (e.g. water soluble).

In various embodiments of the curable mixture, water soluble monomers are dissolved and/or dispersed in an aqueous carrier such as water, or a buffer. In some embodiments, the aqueous carrier comprises a buffer that is selected to maintain the pH in the range of about 7.0 to about 8.4. In other embodiments, the buffer is selected to maintain the pH in the range of about 7.2 to about 8.2, or in the range of about 7.4 to about 8.0. In some embodiments, a base, such as calcium hydroxide, sodium hydroxide and/or lithium hydroxide may be added in an amount to achieve homogeneity or increase solubility of the monomer and/or radiopaque material in the curable mixture, such as about 0.1 wt % to about 5 wt %, based on the total weight of the curable mixture. In some embodiments, the curable mixture or one part of the curable mixture comprises between 10 wt % and 65 wt %, or between 10 wt % and 60 wt %, or between 15 wt % and 65 wt %, or between 15 wt % and 60 wt %, or between 15 wt % and 50 wt %, or between 15 wt % and 25 wt %, or between 20 wt % and 60 wt %, or between 24 wt % and 60 wt %, or between 20 wt % and 65 wt %, or between 25 wt % and 65 wt %, or between 30 wt % and wt %, or between 35 wt % and 65 wt %, or between 40 wt % and 65 wt %, or between 45 wt % and 65 wt %, of the aqueous carrier. In some embodiments, the curable mixture or one part of the curable mixture comprises, or comprises about, 5 wt %, 10 wt %, 15 wt %, 20 wt %, 24 wt %, wt %, 30 wt %, 40 wt %, 45 wt %, 50 wt %, 55 wt %, 60 wt %, 65 wt %, 70 wt %, 75 wt %, 80 wt %, 85 wt %, 90 wt %, 92 wt %, 93 wt %, 95 wt %, 98 wt % or 100 wt % of the aqueous carrier, or any range of values therebetween.

Free-radical polymerization initiators suitable for use in the curable mixtures described herein include a halogen molecule, azo compound, organic peroxide, an inorganic peroxide, or other free-radical polymerization initiators. An example of a halogen molecule is C12, which forms two radicals upon irradiation with ultraviolet light (UV). An azo polymerization initiator may include, but is not limited to, a diazo free radical initiator such as 2,2′-azobis (2-methylpropionamidine) dihydrochloride (AAPH), 2,2′-azobis[2-(2-imidazolin-2-yl)propane] dihydrochloride (AIPH), azobisisobutyronitrile (AIBN) and 1,1′-azobis-(cyclohexanecarbonitrile) (known as ABCN or ACHN), which yield isobutyronitrile and cyclohexanecarbonitrile radicals, respectively, for example, when heated and/or UV irradiated. Examples of organic peroxides include di-tert-butyl peroxide (tBuOOtBu), which forms t-butoxy radicals when heated and/or UV irradiated, and cumene hydroperoxide (CHP). Examples of inorganic peroxides include peroxydisulfate salts such as potassium persulfate (KPS). Free-radical polymerization co-initiators suitable for use herein may include thiosinamine, N-phenylglycine, 2-pyrrolidinone, dimethylaminoethyl acrylate (DMAEA), triethanolamine (TEOA), 1-vinyl-2-pyrrolidone and L-arginine. In some embodiments, the free radical initiator may be present in about 0.1 wt % to about 3 wt %, or about 0.1 wt % to about 2.5 wt %, or about wt % to about 3 wt %, or about 0.5 wt % to about 2 wt %, or 0.2 wt % to about 1.0 wt % based on the total weight of the curable mixture or one part of the curable mixture. In other embodiments, the reaction mixture or one part of the reaction mixture may comprise about 0.2 wt % to 6 wt %, 0.5 wt % to about 3 wt %, or about 0.5 wt % to about 2 wt %, or about 0.2 wt % to 1.5 wt % of a co-initiator. In some embodiments, the free radical initiator may be present in, or in about, 0.1 wt %, 0.2 wt %, 0.5 wt %, 1 wt %, 1.5 wt %, 2 wt %, 2.5 wt %, 3 wt %, 6 wt % or 10 wt % based on the total weight of the curable mixture or one part of the curable mixture, or any range of values therebetween. In other embodiments, the reaction mixture or one part of the reaction mixture may comprise, or comprise about, 0.1 wt %, 0.2 wt %, 0.5 wt %, 1 wt %, 1.5 wt %, 2 wt %, 3 wt %, 4 wt %, 6 wt % or 10 wt % of a co-initiator, or any range of values therebetween.

In an embodiment of a two-part, self-curing or chemical curing composition, a free-radical polymerization initiator and co-initiator are each included in an amount effective to polymerize the acrylate-based, acrylamide-based and/or water-soluble chelating monomer in the curable mixture to form a cured mixture having properties suitable for use as a tooth filling. The curable mixture may polymerize in the range of about 20° C. to about 40° C., thus allowing convenient curing at temperatures in the range of about room temperature to about physiological temperature.

In some embodiments, a heat curable mixture comprising a free-radical polymerization initiator, such as 2,2′-azobis[2-(2-imidazolin-2-yl) propane] dihydrochloride (AIPH/VA-044) is polymerizable around physiological temperature, thus allowing the mixture to cure in situ upon injection into the tooth space. In a further embodiment, light curable compositions are polymerizble, for example, upon exposure to a dental curing light (e.g., 3M Elipar™), with light energy at a λmax wavelength between 400 nm and 500 nm. Light initiators suitable for use herein include, but are not limited to, 2,2′-azobis[2-methyl-N-(2-hydroxyethyl)propionamide (AMPH/VA-086), camphorquinone (CQ), 1-phenyl-1,2-propanedione (PPD), 7,7-dimethyl-2,3-dioxobicyclo[2.2.1] heptane-1-carboxylic acid (CCQ), and mixtures thereof. Light co-initiators may include, but are not limited to N-phenylglycine, 2-pyrrolidinone, dimethylaminoethyl acrylate (DMAEA), 1-vinyl-2-pyrrolidone, triethanolamine (TEOA), L-arginine, and mixtures thereof.

In some embodiments, a dual curable mixture is provided that comprises both a light initiator and a heat initiator that may be cured through sequential exposure to light energy and heat energy. In one embodiment, after filling a tooth space the dual curable mixture may be partial cured upon exposure to light energy, for example, to prevent or reduce movement of a low viscosity material out of the tooth. However, where the light energy may not penetrate beyond a certain depth or into nonlinear or side canals, the obturation material may be further cured through exposure to heat, such as physiological temperature.

A method is provided for forming a cured mixture within a tooth space by positioning a curable mixture within the tooth space, initiating a first curing reaction by exposing the curable mixture to a first energy source to render the curable mixture substantially stable, and initiating a second curing reaction by exposing the stable curable mixture to a second energy source to form the cured material.

In some embodiments, the curable mixture further comprises a polymerization cross-linker. Various polymerization cross-linkers are suitable for use in the curable mixture of ingredients. In some embodiments, the polymerization cross-linker is an acrylate monomer, a polyacrylate monomer, a polymethacrylate ester monomer, or a mixture thereof. In some embodiments, the polymerization cross-linker is of a low molecular weight relative to the water-soluble acrylate-based monomer. Examples include N,N′-methylenebis(acrylamide) (MBAA), triethylene glycol dimethacrylate (TEGDMA), PEG diacrylate (e.g., with Mn 200), ethylene glycol diacrylate, triethylene glycol-bis-methacrylate, ethylene glycol-dimethacrylate, triethyleneglycol-bis-acrylate, 3,3′-ethylidene-bis (N-vinyl-2-pyrrolidone), trimethylolpropane trimethacrylate, pentaerythritol triacrylate, glycerol trimethacrylate, polyethylene glycol dimethacrylate, trimethylolpropane ethoxylate triacrylate, di(trimethylolpropane) tetraacrylate, pentaerythritol tetraacrylate, star shaped 6-arm (TP) PEG-acrylate, multi-arm poly(ethylene glycol) (meth)acrylate, such as 3-arm poly(ethylene glycol) acrylate, 4-arm poly(ethylene glycol) acrylate, 3-arm poly(ethylene glycol) methacrylate, 8-arm poly(ethylene glycol) acrylate, 8-arm poly(ethylene glycol) methacrylate, or mixtures thereof. In some embodiments, 8-arm PEG (meth)acrylate may comprise one or more functional arms and a core comprising, for example hexaglycerol or tripentaerythritol, for example, 8-arm (TP) PEG acrylate (e.g., 8-Arm poly(ethylene glycol) acrylate with tripentaerythritol core. In other embodiments, 3-arm poly(ethylene glycol) (meth)acrylate, may comprise a glycerol or trimethylolpropane core, and one, two or three functional arms.

In some embodiments, the amount of polymerization cross-linker is, or is approximately 0.005 wt. %, 0.01 wt. %, 0.02 wt. %, 0.03 wt. %, 0.04 wt. %, 0.05 wt. %, 0.06 wt. %, wt. %, 0.1 wt. %, 0.12 wt. %, 0.15 wt. %, 0.2 wt. %, 0.3 wt. %, 0.5 wt. %, 0.8 wt. %, 1 wt. %, 1.5 wt. %, 2 wt. %, 2.5 wt. %, 3 wt. % or 5 wt. % of the total weight of the mixture, or any range of values therebetween. For example, in some embodiments the amount of polymerization cross-linker is, or is approximately, 0.02 wt. % to 2 wt. % of the total weight of the mixture, or 0.05 wt. % to 1 wt. %, or 0.5 wt. % to 5 wt. %, of the total weight of the mixture. In some embodiments, the amount of polymerization cross-linker is, or is approximately 0.05 wt. %, 0.06 wt. %, 0.08 wt. %, wt. %, 0.12 wt. %, 0.15 wt. %, 0.2 wt. %, 0.3 wt. %, 0.5 wt. %, 0.8 wt. %, 1 wt. %, 1.5 wt. %, 2 wt. %, 2.5 wt. %, 3 wt. %, 4 wt. %, 5 wt. %, 6 wt. %, 7 wt. %, 8 wt. %, 10 wt. %, 12 wt. %, 15 wt. % or wt. % of the total amount of monomer (i.e. acrylate-based monomer, acrylamide-based monomer, chelating monomer and polymerization cross-linker), or any range of values therebetween. For example, in some embodiments the amount of polymerization cross-linker is, or is approximately, 0.1 wt. % to 10 wt. % of the total amount of monomer, or 1 wt. % to 8 wt. % of the total amount of monomer. In some embodiments, the weight ratio of polymerization cross-linker to monomer is, or is approximately, 1:100, 1:75, 1:60, 1:50, 1:40, 1:30, 1:20, 1:15, 1:10 or 1:5, or any range of values therebetween. For example, in some embodiments the weight ratio of polymerization cross-linker to total amount of monomer is, or is approximately, 1:50 to 1:10, or 1:30 to 1:15.

In some embodiments, the curable mixture further comprises an acid, such as an acid monomer, suitable for use in the curable mixture. In some embodiments, the acid monomer is an acrylic acid. Examples include methacrylic acid, acrylic acid, methacryloyloxyethyl succinate, or mixtures thereof. Acid monomers that increase the hydrophilicity and/or provide crosslinking sites may be suitable for use in making hydrogel polymers for use as obturation materials. In some embodiments, the amount of the acid monomer may be between 0.1 wt % and 0.5 wt %, or between 0.2 wt % and 0.3 wt %, based on the total weight of the curable mixture. In some embodiments, the amount of the acid monomer may be, or be about, 0.1 wt %, 0.2 wt %, 0.3 wt %, 0.4 wt %, 0.5 wt %, 0.7 wt % or 1 wt % based on the total weight of the curable mixture, or any range of values therebetween.

In some embodiments, the curable mixture further comprises an antimicrobial or antibacterial reagent. Various antimicrobial reagents are suitable for use in the curable mixture of ingredients. Examples include, but are not limited to, zinc oxide, (3-acrylamidolpropyl)trimethyl-ammonium chloride (APTA), [2-(acryloyloxy)ethyl]trimethyl-ammonium chloride (EGAA), or mixtures thereof. When present, the amount of the antimicrobial monomer may be between 0.1 wt % and 2.5 wt %, based on the total weight of the curable mixture.

In some embodiments, obturation materials formed from curable materials disclosed herein may reduce the amount of harmful bacteria for Escherichia coli (E. coli). Enterococcus faecalis (E. faecalis), or both. In some embodiments, materials described herein may demonstrate at least a 1 log reduction, or at least a 2 log reduction, from the initial count of E. coli, E. faecalis, or both, when tested in an initial biofilm study according to USP <51> Antimicrobial Effectiveness Testing.

In some embodiments, the curable mixture further comprises a surfactant. Various surfactants are suitable for use in the curable mixture of ingredients. Examples include Triton X-100.

In some embodiments, the curable mixture further comprises an inhibitor. Various inhibitors are suitable for use in the curable mixture of ingredients. Examples include 4-methoxyphenol (MEHQ), hydroquinone (HQ) and 2,6-di-tert-butyl-4-methyl phenol (BHT). In various embodiments the inhibitor is used in an amount effective to control the curing time of the curable mixture.

In various embodiments, at least a portion of the curing of the curable mixture takes place after positioning the curable mixture in a cavity or root canal. For example, an embodiment provides a method of filling a tooth, comprising identifying a tooth having a cavity in need of filling; positioning a curable mixture as described herein within the cavity; and curing the curable mixture within the cavity. Another embodiment provides a method of filling a tooth, comprising identifying a tooth having a root canal in need of filling; positioning a curable mixture as described herein within the root canal; and curing the curable mixture within the root canal. The positioning of the curable mixture in the cavity or root canal can be carried out in various ways as described elsewhere herein. In a further embodiment, a method comprises providing a curable obturation material in at least two parts, wherein each part of the curable material is a liquid that is delivered separately to a handheld device, for example, through first and second ports of the handheld device. In an embodiment, the handheld device comprises a liquid jet device for forming liquid jet comprising first and/or second solutions of a two-part curable obturation material that are provided through first and second ports of the handheld device. The first and second parts are then mixed to form a curable obturation material by the liquid jet device prior to filling a tooth space, wherein the curable obturation material cures to form a solid hydrogel.

Optionally, the curable obturation material is degassed prior to mixing the first and second solutions. The percent reduction of dissolved gas (for example, mg/L dissolved oxygen) may be at least 10% after degassing. Optionally, one or more of a first part, second part and/or the curable obturation reaction mixture have a viscosity of less than 100 cP, less than 60 cP, or less than 40 cP, or less than 30 cP or less than 20 cP, at ambient conditions (approximately 25° C.), for example, when measured on a Brookfield viscometer.

To protect the root canal from infection over time, an obturation material may be resistant towards degradation. In some embodiments, a durable cured obturation material comprises hydrogel having a hardness greater than 15 Shore A, a hardness greater than 20 Shore A, or greater than 60 Shore A, or greater than 70 Shore A, or between 15 Shore A and 90 Shore A, or between 20 Shore A and 90 Shore A. Hydrogel obturation materials may exhibit low volumetric shrinkage upon curing and low diametral swelling upon curing. In one embodiment, cured obturation materials provided herein have a diametral swelling is less than 40%.

A curable obturation material may further comprise calcium silicate to enhance durability of the cured obturation material. In this embodiment, the curable obturation material may comprise (a) a water soluble acrylate-based monomer, a water-soluble acrylamide-based monomer, a water-soluble chelating monomer, or a mixture thereof; (b) a calcium silicate compound; (c) a low viscosity liquid composition; and (d) a free radial polymerization initiator. Optionally, a crosslinker, co-initiator, water soluble radiopaque agent and/or an antimicrobial compound, such as quaternary ammonium salt, as described above for hydrogel mixtures may be included.

Water soluble monomers for use with calcium silicate may include at least one acrylate-based monomer, acrylamide-based monomer, chelating monomer, or mixtures thereof, as provided throughout the disclosure and examples. A low viscosity carrier liquid may have a viscosity of about 1 cps to about 30 cps at 25° C. In some embodiments, the low viscosity carrier liquid has a viscosity between about 0.1 cps and 20 cps at 25° C. The low viscosity liquid may comprise an aqueous liquid provided herein, or a non-aqueous carrier liquid, for example as provided above for use as a carrier liquid for calcium silicate. Various calcium silicate compounds are suitable for use in the curable mixture of ingredients. In some embodiments, the calcium silicate compound comprises at least one of calcium silicate, dicalcium silicate, or tricalcium silicate. In some embodiments, the calcium silicate component consists essentially of tricalcium silicate.

In one aspect, the components of the curable obturation material may be provided in at least two liquid parts. In one aspect, a three-part system for forming a curable obturation material contains, Part (A) that comprises a water soluble acrylate-based monomer, a water-soluble acrylamide-based monomer, a water-soluble chelating monomer, or a mixture thereof, and an aqueous or non-aqueous solvent; Part (B) that comprises a calcium silicate compound and a non-aqueous carrier liquid; and Part (C) that comprises a low viscosity liquid composition. Parts (A), (B) and (C) may be combined in a weight ratio in a range, such as, from 1 to 3 part A: 1 to 3 part B: 0.1 to 1 part C. A free radical polymerization initiator, and optional components, including but not limited, to a water soluble radiopaque material, a co-initiator and/or crosslinker may be mixed in one or more of Parts (A), (B) or (C).

A curable obturation material may contain various amounts of the calcium silicate compound. For example, in some embodiments, the amount of the calcium silicate compound (e.g. tricalcium silicate) in the curable mixture is in any one of the following ranges: 1 wt. % to 40 wt. %, 1 wt. % to 30 wt. %, 1 wt. % to 20 wt. %, 1 wt. % to 18 wt. %, 1 wt. % to 15 wt. %, 1 wt. % to 13 wt. %, 1 wt. % to 12 wt. %, 1 wt. % to 10 wt. %, 5 wt. % to 15 wt. %, 5 wt. % to 13 wt. %, 5 wt. % to 12 wt. %, 5 wt. % to 10 wt. %, 7 wt. % to 15 wt. %, 7 wt. % to 13 wt. %, 7 wt. % to 12 wt. %, 7 wt. % to 10 wt. %, 8 wt. % to 15 wt. %, 8 wt. % to 13 wt. %, 8 wt. % to 12 wt. %, 8 wt. % to 10 wt. %, 10 wt. % to 20 wt. %, 10 wt. % to 18 wt. %, 10 wt. % to 15 wt. %, or 10 wt. % to 13 wt. %, based on total weight of curable mixture. In some embodiments, the curable mixture comprises the calcium silicate compound (e.g. tricalcium silicate) in any one of the amounts within the aforementioned ranges, such as, or about, 1 wt. %, 3 wt. %, 5 wt. %, 7 wt. %, 8 wt. %, 9 wt. %, 10 wt. %, 11 wt. %, 12 wt. %, 13 wt. %, 14 wt. %, 15 wt. %, 16 wt. %, 17 wt. %, 18 wt. %, 19 wt. %, or 20 wt. %, or 30 wt. %, or 40 wt. %, or any range of values therebetween. In some embodiments, the calcium silicate compound is in a microparticulate form. In some embodiments, the microparticles have an average particle size of about 5 microns or less, about 3 microns or less, or about 2 microns or less. In some embodiments, the calcium silicate compound is substantially anhydrous.

In one aspect, Part (B) comprising a calcium silicate compound and non-aqueous carrier liquid may comprise at least one of acetic acid, acetone, acetonitrile, 1-butanol, 2-butanone, ethyl acetate, methanol, ethanol, propanol, butanol, dimethyl sulfoxide, dimethylformamide, 1,4-dioxane, methyl isocyanide, pyridine, tetrahydrofuran, ethylene glycol, propylene glycol, triethylene glycol, poly(ethylene glycol), poly(propylene glycol) and glycerol. In some embodiments, the carrier liquid comprises at least one of acetic acid, 1-butanol, methanol, ethanol, propanol, butanol, dimethyl sulfoxide, dimethylformamide, ethylene glycol, propylene glycol, triethylene glycol, poly(ethylene glycol), poly(ethylene glycol) (meth)acrylate, poly(propylene glycol), glycerol and diethylene glycol monomethyl ether. In some embodiments, the non-aqueous carrier liquid is water soluble or water miscible. In some embodiments, the carrier liquid is substantially anhydrous.

In some embodiments, the non-aqueous carrier liquid of Part (B) may be selected to form flowable paste with the calcium silicate, the paste having a flow rate of at least about 20 grams per minute (g/min) at 20 psi when measured, for example, by the Paste Flow Rate test method provided herein. In some embodiments, Part (B) has a flow rate in the range of about 20 g/min. to about 1000 g/min., or about 50 g/min. to about 500 g/min., or about 100 g/min. to about 500 g/min., or about 100 g/min. to about 400 g/min., at 20 psi. In some embodiments, the flow rate is in the range of about 50 g/min. to about 500 g/min. for at least 4 days. In some embodiments, the curable mixture comprises 1 wt % to 20 wt % tricalcium silicate, or 1 wt % to 15 wt % tricalcium silicate, or 5 wt % to 15 wt % tricalcium silicate, or 7 wt % to 13 wt % tricalcium silicate. In some embodiments, the calcium silicate consists essentially of tricalcium silicate.

When mixed together, the three-part mixture forms a curable or polymerizable restorative composition that is cured or hardened to form the final material. In some embodiments, curing is initiated upon mixing at least two of parts (A), (B) and (C). The curable mixture may polymerize, for example, in the range of about 20° C. to about 40° C., thus allowing convenient curing at temperatures in the range of about room temperature to about physiological temperature. The three-part system may further comprise an x-ray contrast agent, such as a water soluble radiopaque material, and/or an antimicrobial material such as an acrylate monomer that contains a quaternary ammonium group, as described above for the curable hydrogel mixtures.

In some embodiments, the first part, second part and third part of the three-part curable mixture are combined prior to the mixture being introduced into the space or spaces of a tooth. The components of the mixture also, may be introduced into spaces of the tooth. Parts A, B and C may be delivered to a patient by way of an application device, for example, as described below. In one aspect, an application delivery device optionally delivers a low viscosity component such as Part C, through the device nozzle, and two other parts, such as Parts A and B, which may have higher viscosities, may be delivered through side-ports. In some embodiments, the three-part curable mixture is provided as a kit that comprises a first container comprising the first part of the mixture, a second container comprising the second part of the mixture and the third container comprising a third part of the mixture.

### Application Device

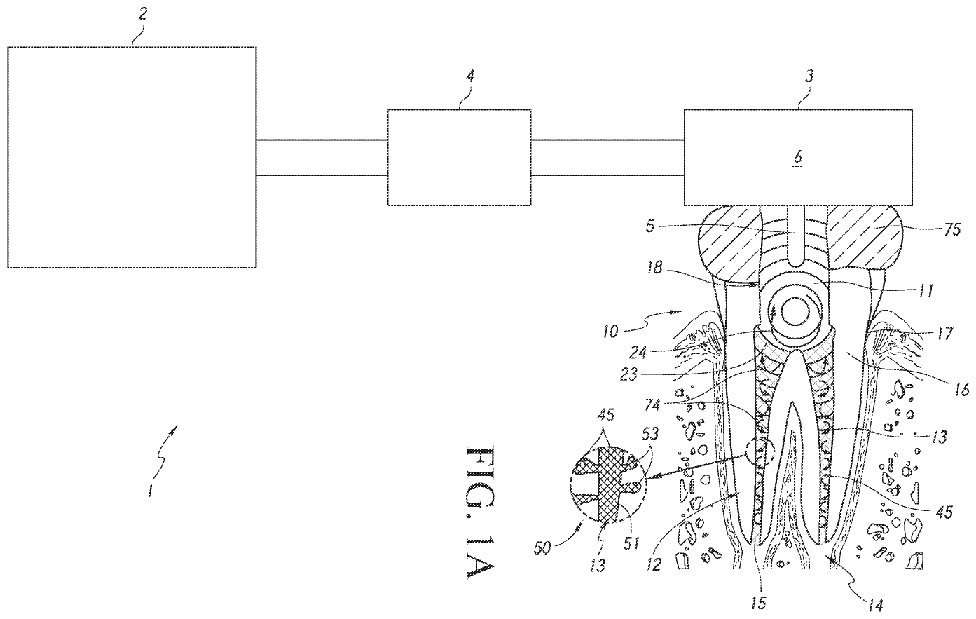

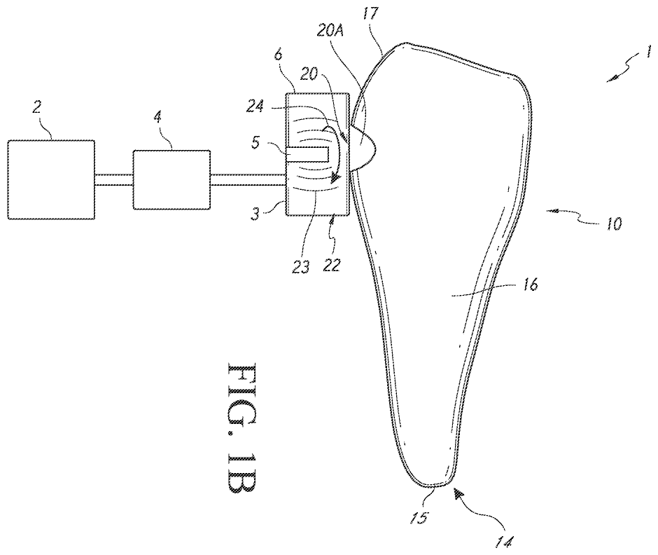

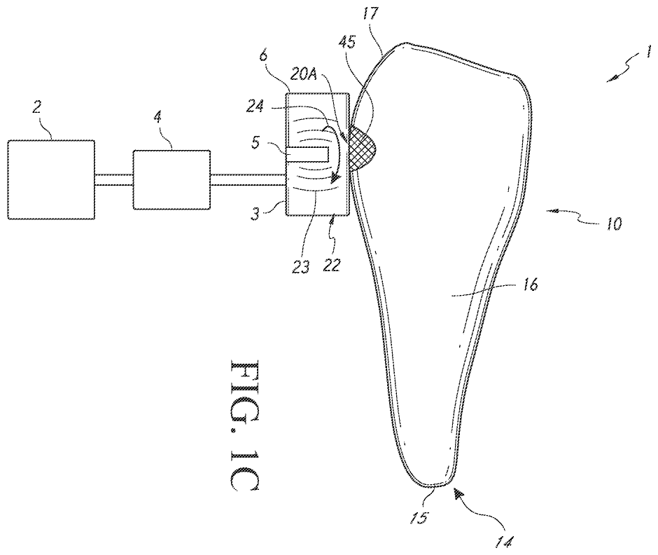

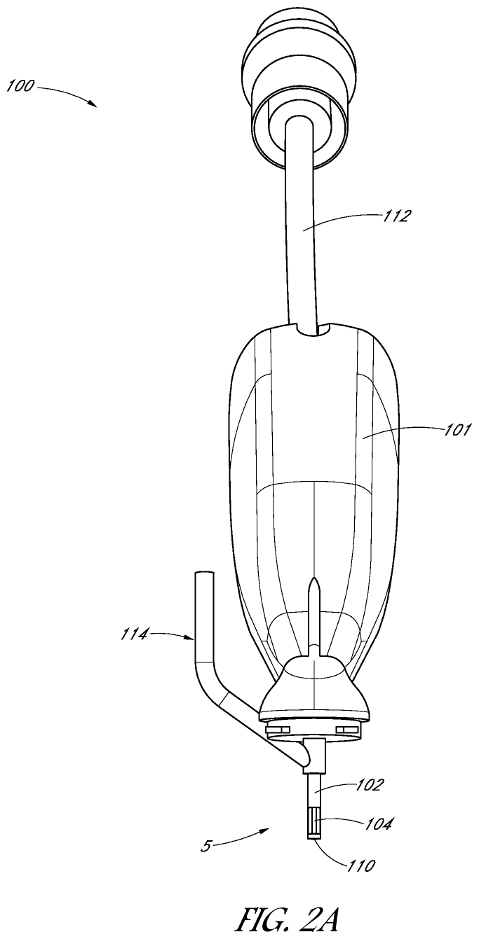

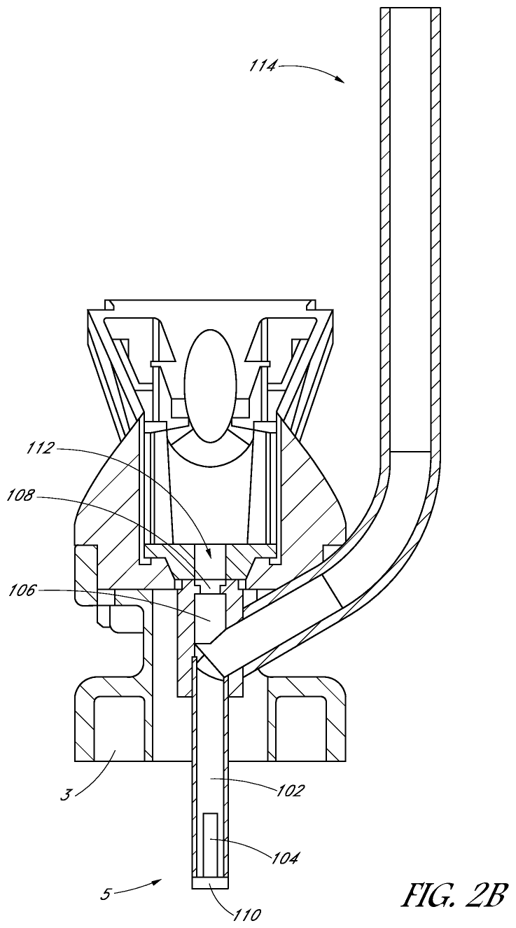

The curable obturation materials and cured obturation materials described herein may be applied to a tooth by various methods and devices. The filling or obturation material may be formed in any suitable manner. For example, in some embodiments, a clinician can form the obturation material by mixing the obturation material ingredients, e.g., by hand, by a mechanical tool, or by a mixing device. Furthermore, the obturation material can be applied to a tooth in any suitable manner. For example, in some embodiments, a clinician may apply the obturation material in the tooth, e.g., by hand, syringe, mechanical tool, or application device. In FIGS. 1 through 2B, embodiments of a mixing device and/or an application device that can be used to form and/or apply an obturation material are disclosed. In some embodiments, a clinician can form the obturation material by mixing the obturation material ingredients outside of an application device, place the obturation material into an application device, and apply the obturation material to a tooth using the application device. A composition consisting of all the ingredients of the curable mixture except for at least one missing ingredient may be loaded into an application device, and the composition and the missing ingredient may be combined within the application device to form the obturation material, and the obturation material is applied to a tooth using the application device.

FIG. 1A is a schematic diagram of a system 1, in accordance with embodiments of an application or delivery device as disclosed herein. The system 1 can be configured to perform various types of treatment procedures, including, e.g., cleaning treatments, obturation or other filling treatments, restoration treatments, etc. In the embodiment shown in FIG. 1A, the system 1 is illustrated as being coupled to (e.g., positioned against in some arrangements) a tooth 10 that is a molar tooth of a mammal, such as a human. However, the tooth 10 can be any other suitable type of tooth, such as a pre-molar, bicuspid, incisor, canine, etc. Furthermore, the system 1 shown in FIG. 1A can include components configured to remove unhealthy or undesirable materials from a tooth or surrounding gum tissue, for example, a root canal 13 of the tooth 10. Thus, in the embodiment of FIG. 1A, the system 10 can also be configured to clean the tooth 10, in addition to being configured to fill or obturate the tooth. Moreover, although the treatment shown in FIG. 1A is a root canal treatment, in other embodiments, the application device and obturation material(s) disclosed herein can be used to fill other types of treatment regions, such as a treated carious region of the tooth.

The tooth 10 includes hard structural and protective layers, including a hard layer of dentin 16 and a very hard outer layer of enamel 17. A pulp cavity 11 is defined within the dentin 16. The pulp cavity 11 comprises one or more root canals 13 extending toward an apex 14 of each root 12. The pulp cavity 11 and root canal 13 contain dental pulp, which is a soft, vascular tissue comprising nerves, blood vessels, connective tissue, odontoblasts, and other tissue and cellular components. Blood vessels and nerves enter/exit the root canal 13 through a tiny opening, the apical foramen or apical opening 15, near a tip of the apex 14 of the root 12. It should be appreciated that, although the tooth 10 illustrated herein is a molar, the embodiments disclosed herein can advantageously be used to treat any suitable type of tooth, including pre-molars, canines, incisors, etc.

The system 1 can include a console 2, a pressure wave generator 5, and a tooth coupler 3 (such as a handpiece) adapted to couple to the tooth 10. The tooth coupler 3 can couple to the tooth 10 in any suitable way. In some arrangements, the tooth coupler 3 can be positioned against and/or attach to the tooth 10 by way of a tooth seal 75. For example, the clinician can hold the tooth coupler 3 against the tooth 10 during treatment. In some embodiments, the tooth coupler 3 can define a chamber 6 configured to retain fluid therein, such as a filler or obturation material described herein. In some embodiments, the pulp cavity 11 can define a tooth chamber configured to retain fluid therein. In some embodiments, the tooth coupler 3 may not define a chamber, and the tooth chamber defined at least in part by the pulp cavity 11 can retain fluid.

The tooth coupler 3 disclosed herein can be any suitable structure or housing configured to couple to the tooth 10 for a treatment procedure. As used herein, “couple” is meant to include arrangements in which there is a connection with the tooth 10, as well as arrangements in which the coupler 3 is placed against or in the tooth and is held by the clinician in that position. The pressure wave generator 5 can be coupled to and/or disposed in or on the tooth coupler 3 in various embodiments.

A system interface member 4 can electrically, mechanically, and/or fluidly connect the console 2 with the tooth coupler 3 and pressure wave generator 5. For example, in some embodiments, the system interface member 4 can removably couple the tooth coupler 3 to the console 2. In such embodiments, the clinician can use the tooth coupler 3 one time (or a few times) and dispose of the tooth coupler 3 after each procedure (or after a set number of procedures). The console 2 and interface member 4 can be reused multiple times to removably couple (e.g., to connect and/or disconnect) to multiple tooth couplers 3 using suitable engagement features, as discussed herein. The interface member 4 can include various electrical and/or fluidic pathways to provide electrical, electronic, and/or fluidic communication between the console 2 and the tooth coupler 3. The console 2 can include a control system and various fluid and/or electrical systems configured to operate the pressure wave generator 5 during a treatment procedure. The console 2 can also include a management module configured to manage data regarding the treatment procedure. The console 2 can include a communications module configured to communicate with external entities about the treatment procedures. Additionally, the console 2 can include a control system comprising a processor and non-transitory memory. Computer-implemented instructions can be stored on the memory and can be executed by the processor to assist in controlling cleaning and/or filling procedures. Additional details of the console 2 can be found in U.S. Pat. No. 9,504,536, and in U.S. Pat. No. 9,675,426, each of which is incorporated by reference herein in its entirety and for all purposes.

In FIG. 1A, the system 1 is used to fill or obturate the root canal 13 with an obturation material 45, which can be the same as, or generally similar to, the filler materials described herein. When the root canal 13 is cleaned, the clinician can supply an obturation material 45 in a flowable state to the pulp cavity 11, canals 13, or other internal chambers of the tooth 10. In some embodiments, a pressure wave generator 5 may be coupled to, or formed with, a handpiece having one or more openings configured to deliver the flowable obturation material to the tooth 10. In still other embodiments, a dental handpiece may include one or more supply lines to supply the flowable obturation material 45 to the tooth 10. An obturation material may have a first state which is flowable, to flow through the treatment region to fill the root canals 13 and/or pulp cavity 11. The obturation material 45 may harden to form a second state by solidifying after filling the treatment region.

Advantageously, the pressure wave generator 5 can be activated to assist in positioning the obturation material 45 throughout the treatment region to be filled, thereby assisting in substantially filling the tooth 10. As shown in inset 50 of FIG. 1A, for example, when activated, the pressure wave generator 5 may cause the obturation material 45 to flow into major canal spaces 51 of the tooth 10, as well as into small spaces 53 of the tooth 10. Thus, the system 1 shown in FIG. 1A can assist in filling small cracks, tubules, and other tiny spaces (e.g., the small spaces 53) of the tooth 10. By filling the small spaces 53 of the tooth, the system 1 can ensure a more robust obturation procedure which results in long-term health benefits for the patient. The pressure waves 23 and/or fluid motion 24 (which can include vortices 74) generated by the pressure wave generator 5 can interact with the obturation material 45 to assist in filling the small spaces 53 and the major spaces 51 of the tooth 10. Furthermore, in some embodiments, the pressure wave generator 5 can be activated to assist in curing or hardening the obturation material 45. In some embodiments, curing or hardening of the obturation materials may be enhanced when agitated by pressure waves 23 generated by the pressure wave generator 5.

Obturation or filling material may be degas sed to facilitate delivery of the obturation material to spaces of the tooth. In some embodiments, presence of dissolved gas in an obturation material may result in bubbles that block, or inhibit, the transfer or flow of, the obturation material and/or pressure waves into spaces within a tooth. Upon curing the obturation material, portions of a tooth space or root canal that have not been filled with obturation material may be visualized, for example, by conventional dental X-ray analysis. Where the one or more parts of a reaction mixture solution, or the reaction mixture solution itself, are degassed prior to penetration into the tooth, fewer bubbles may come out of a solution, and a degassed composition may substantially and/or completely penetrate non-linear or small spaces, for example, having a diameter smaller than 500 microns, or smaller than 100 microns, or smaller than 10 microns. In some embodiments, the degassed composition may substantially and/or completely penetrate non-linear or small spaces having a diameter of, of about, less than, or less than about, 1000 microns, 500 microns, 100 microns or 10 microns, or any range of values therebetween.

As used herein, a degassed composition has a dissolved gas content that has been reduced after a degassing step. In some embodiments, dissolved gas content may be reduced by approximately 1% to 70%, or reduced by 5% to 50%, or reduced by 10% to 40%. In some embodiments, dissolved gas content may be reduced by, or by approximately, 1%, 5%, 10%, 20%, 40%, 50%, 70% or 80%, or any range of values therebetween. In some embodiments, the amount of dissolved gas in the liquid compositions before or after degassing may be measured in terms of the amount of dissolved oxygen (e.g., mg/L), for example, by titration, or optical or electrochemical sensors that perform a dissolved gas analysis, such as Pro-Oceanus GTD-Pro or HGTD dissolved gas sensor available from Pro-Oceanus Systems Inc. (Nova Scotia, Canada), or D-Opto dissolved oxygen sensor available from Zebra-Tech Ltd. (Nelson, New Zealand). A degassing step may include known degassing techniques or combinations of thereof, such as heating, helium sparging, vacuum, filtering, de-bubbling, sonication, and the like. In one embodiment, after degassing the curable reaction mixture has an oxygen concentration of 0 mg/L to 3.2 mg/L, when measured using a dissolved oxygen meter.

In some embodiments, the obturation material 45 is supplied to the tooth 10, and the pressure wave generator 5 is subsequently activated to enhance the obturation procedure (e.g., to improve the filling process and/or to enhance or activate the curing process). Sequentially, the clinician may supply the obturation material 45 to the tooth 10 using a syringe or other device, and the pressure wave generator 5 may subsequently (or concurrently) be activated to fill the treatment region. In other embodiments, the pressure wave generator 5 may supply the obturation material 45 and generate pressure waves through the obturation material (or other fluids at the treatment region) simultaneously, or the steps of supplying the obturation material to the tooth and filling the tooth by generating pressure waves within the tooth, may overlap in time. For example, where the pressure wave generator 5 comprises a liquid jet device, one or more components of obturation material may be provided in the treatment region as a liquid jet to enhance the obturation procedure.

As disclosed herein, a pressure wave generator 5 comprises any suitable wave generator, including but not limited to a liquid jet device, a laser, a mechanical stirrer, and an ultrasonic transducer. The pressure wave generator 5 may be disposed outside the region of the tooth 10, having a chamber 6 disposed outside the tooth 10. In other arrangements, a pressure wave generator 5 extends partially into the tooth 10. In some arrangements, the pressure wave generator 5 can extend to a depth that does not interfere with the filling. The system 1 can include a cleaning mode for cleaning the treatment region and a filling mode to fill or obturate the treatment region. The console 2 can include a control system comprising a processor and memory. The control system can be programmed or configured to switch the system 1 from the cleaning mode to the filling mode and vice versa. The control system of the console 2 can also control the operation of cleaning and/or filling procedures. Additional details of the delivery device shown in FIG. 1A can be found throughout U.S. Pat. No. 9,877,801, the entire contents of which are incorporated herein by reference and particularly for the purpose of describing such details.

FIG. 1B is a schematic diagram of a system 1 that includes components configured to clean unhealthy or undesirable material from a treatment region 20 on an exterior surface of the tooth 10. For example, as in FIG. 1A, the system 1 can include a tooth coupler 3 and a pressure wave generator 5. The tooth coupler 3 can communicate with a console 2 by way of a system interface member 4. Unlike the system 1 of FIG. 1A, however, the tooth coupler 3 is coupled to (e.g., positioned against by a clinician) a treatment region 20 on an exterior surface of the tooth 10. In some embodiments, the tooth coupler 3 can be stably positioned against the treatment region and can be sealed to the tooth 10, e.g., by way of an adhesive or other seal. The system 1 of FIG. 1B can be activated to clean an exterior surface of the tooth 10, e.g., a carious region of the tooth 10 and/or remove undesirable dental deposits, such as plaque, calculus biofilms, bacteria, etc, from the tooth 10 and/or surround gum tissue. In other embodiments (e.g., FIG. 1C), the system 1 can be activated to fill a treated region on the exterior surface of the tooth 10 with a filling or restoration material. As with the embodiment of FIG. 1A, pressure waves 23 and/or fluid motion 24 can be generated in the tooth coupler 3 and chamber 6, which can act to clean the treatment region 20 of the tooth 10, forming a cleaned treatment region 20A in which the carious (or other unhealthy material) is removed. Additional details of systems and methods for treating carious regions of teeth can be found in International Application Publication WO 2013/142385 (PCT/US2013/032635), having an international filing date of Mar. 15, 2013, entitled “APPARATUS AND METHODS FOR CLEANING TEETH,” the entire contents of which are incorporated by reference herein in their entirety and for all purposes. Additional details of systems and methods for removing undesirable dental deposits (such as plaque, calculus, etc.) from teeth and/or gums can be found in International Application Publication WO 2013/155492 (Application No. PCT/US2013/036493), having an international filing date of Apr. 12, 2013, entitled “APPARATUS AND METHODS FOR CLEANING TEETH AND GINGIVAL POCKETS,” and in U.S. Patent Publication No. US 2014/0099597, filed Apr. 11, 2013, entitled “APPARATUS AND METHODS FOR CLEANING TEETH AND GINGIVAL POCKETS,” each of which is incorporated by reference herein in its entirety and for all purposes.

FIG. 1C is a schematic diagram of the system 1 of FIG. 1B, in which the system 1 is configured to fill the treated carious region 20A of the tooth 10, and can be used in combination with any of the filling materials disclosed herein. As with the embodiment of FIG. 1B, the system can include a pressure wave generator 5, a tooth coupler 3, an interface member 4, and a console 2. When the carious or other unhealthy material is removed from the tooth 10, the clinician can fill the cleaned treatment region 20A with a suitable filler or obturation material As with the embodiment of FIG. 1A, the obturation material 45 can be supplied to the cleaned treatment region 20A. The pressure wave generator 5 can act to substantially fill the treatment region 20A and/or to enhance or activate the hardening of the filler obturation material 45. In some embodiments, the filler or obturation material 45 is supplied to the tooth 10, and the pressure wave generator 5 is subsequently activated to enhance the filling procedure (e.g., to improve the filling process and/or to enhance or activate the curing process). For example, in such embodiments, the clinician can supply the filler or obturation material 45 to the treatment region 20A using a syringe, and the pressure wave generator 5 can subsequently be activated to fill the treatment region. In other embodiments, the pressure wave generator 5 is activated to supply the filler or obturation material 45 to the treatment region 20A and to generate pressure waves through the material. For example, in embodiments in which the pressure wave generator comprises a liquid jet device, a jet of obturation or filler material 45 (or other type of fluid) can interact with fluids at the treatment region 20A (e.g., other portions of the filler or obturation material or other treatment fluid) to generate pressure waves that propagates through the fluids. The resulting pressure waves can enhance the obturation procedure.

FIGS. 2A and 2B depict a delivery device 100 that can be used to combine a first composition with a second composition to form the curable mixture and apply it to a treatment region of the tooth to fill the treatment region. As shown in FIGS. 2A-2B, the delivery device 100 can comprise a treatment instrument 101. The treatment instrument 101 can be used to position the pressure wave generator 5 at or near the treatment region. In the embodiment of FIG. 2A, the treatment instrument 101 comprises a handpiece sized and shaped to be held by the clinician against a portion of the tooth. Further, the delivery device 100 can comprise a first composition supply line 112 and a second composition supply line. The first composition supply line 112 can be configured to supply the first composition to a distal portion of the handpiece 101. The second composition supply line 114 can be configured to supply the second composition to the distal portion of the handpiece 101. For example, in some embodiments, the first composition line 112 can be configured to supply the carrier liquid to the tooth, and the second composition 114 can supply other component materials to mix with the carrier liquid.

In FIG. 2A, a pressure wave generator 5 can be coupled to or formed with the distal portion of the handpiece 101. As explained above in connection with FIG. 1A through FIG. 1C, the pressure wave generator 5 can be activated to generate pressure waves and/or fluid motion at the treatment region, to cause the filling or obturation material to fill the treatment region. As explained above, the pressure wave generator 5 can comprise any suitable type of pressure wave generator, including those described in U.S. Pat. No. 9,877,801, the entire contents of which are incorporated herein by reference in their entirety and for all purposes. For example, the pressure wave generator 5 of FIGS. 2A-2B comprises a liquid jet device. The liquid jet device can comprise a nozzle or orifice 108 sized and shaped to pressurize the first composition that is supplied to the orifice 108 by way of the first composition supply line 112. In some embodiments, the orifice 108 can form the first composition into a liquid jet, e.g., a coherent, collimated liquid jet. The liquid jet formed of the first composition can pass into a mixing chamber 106 disposed distal the orifice 108. Thus, in FIG. 2B, the second supply line 114 can be positioned to deliver the second composition to the mixing chamber 106 at a location distal the orifice 108. Thus, the liquid jet of, for example, the carrier material, can be formed and can pass through the mixing chamber 106 to interact with other component materials of a curable obturation material supplied by the second supply line 114.

As shown in FIG. 2B, the second composition supply line 114 can supply the second composition to the mixing chamber 106 by way of one or more ports. The first and second compositions can accordingly be mixed within the mixing chamber 106 to at least partially form the mixed composition of the filling or obturation material. The momentum of the liquid jet can drive the at least partially mixed first and second compositions along a guide tube 102. The liquid jet can impinge on an impingement member 110 located at a distal portion of the guide tube 102. The delivery device 100 can comprise a side port delivery device in which the curable mixture is supplied to the treatment region through one or a plurality of openings 104 in the guide tube 102. The openings 104 can be disposed proximal the impingement member 110. Interaction of the at least partially mixed first and second compositions with fluid in the treatment region can generate pressure waves and/or fluid motion at the treatment region. The pressure waves and/or fluid motion can assist in filling or obturating the treatment region. Additional details of liquid jet devices used for filling a treatment region can be found in FIGS. 4A through 8D of U.S. Pat. No. 9,877,801, the entire contents of which are incorporated by reference herein in their entirety and for all purposes.

Accordingly, in some embodiments, the first and second compositions can be kept separate until combined in the mixing chamber 106 of the delivery device 100 to form the curable mixture. For example, in some embodiments, the first or second composition can consist of all the ingredients of the curable mixture except for at least one missing ingredient. In some embodiments, the missing ingredient can be the carrier fluid or a portion of the carrier fluid, whereby combination of the second composition decreases the viscosity of the first composition in order to create a curable mixture suitable for delivery to the treatment region. In some embodiments, the missing ingredient can initiate curing or hardening of the curable obturation material formed when combining the first and second compositions. In some embodiments, at least one of the first and second compositions are introduced into the curable mixture as a fluid jet as explained herein.

Although the examples shown in FIGS. 1-2B describe the delivery device as including a pressure wave generator, it should be appreciated that the obturation material(s) described herein can be used in conjunction with any other suitable type of delivery device. For example, the obturation material(s) described herein can be delivered to the tooth with a syringe, a mechanical instrument, or any other suitable device.

### Kits

The curable materials, obturation materials and the application devices described herein can be combined in the form of a kit. In some embodiments, the kit includes a first container comprising a first mixture of a composition consisting of all the ingredients of the curable mixture except for at least one missing ingredient, a second container comprising a second mixture of a composition comprising the missing ingredient, and an application device. In one embodiment, a kit for dispensing a curable hydrogel obturation material comprises 1) a curable obturation mixture provided as two liquid parts, wherein a first liquid part comprises a water soluble acrylate-based polymer, and the second liquid part comprises an initiator, and 2) a handpiece for delivering the curable obturation material to a tooth comprising a first opening for receiving the first liquid part, a second opening for receiving the second liquid part, a mixing chamber, and a nozzle to dispense the mixture into a tooth.

In other embodiments, a kit comprises obturation materials as described herein (for example, the first and second containers described above), and not the application devices. In some embodiments, the elements of the kit are packaged together in a single packaging.

### EXAMPLES

### Radiopacity

The determination of radiopacity of compositions was tested by reference to a specimen of an aluminum (Al) standard according to ISO 6876:2012

### Leachable

Leachable testing of the materials was determined according to test 5.6— Solubility per ISO 6876:2012 Root Canal Sealing Materials, and calculated as follows:

$$\% leachable = \frac{\begin{matrix}mass of petri dish after - \\ mass of petri dish before\end{matrix}}{\begin{matrix}mass of sample 1 + \\ mass of sample 2\end{matrix}} * 100$$

### Swelling

Swelling (expansion) was determined by placing material in circular plastic molds with ca. 10 mm diameter and 6 mm thickness. Cured samples were measured, and allowed to condition in water and/or phosphate buffered saline (PBS) for at least 24 hours at 37° C. and calculated as follows:

$$\% expansion = \frac{( \begin{matrix}sample diameter after conditioning - \\ sample diameter initial\end{matrix} )}{sample diameter initial} ⨯ 100$$

Percent water uptake was calculated as follows:

$$\% water uptake = \frac{( \begin{matrix}sample 1 after swelling + \\ sample 2 after swelling\end{matrix} ) - ( \begin{matrix}sample 1 befo re swelling + \\ sample 2 before swelling\end{matrix} )}{2} * 100$$

### Examples 1-7

Hydrogels obturation materials were prepared from aqueous solutions comprising poly(ethylene glycol) diacrylate or trimethacrylate solutions with varying water content The solutions of Examples 1 through 7 further comprised a water soluble radiopaque material.

For each example, all components listed in Table 1 were mixed together to form a one-part solution and cured as described in Table 1. The solutions comprised one or more free-radical initiators and were exposed to light and/or heat. Durability as measured by hardness was assessed.

TABLE 1
Hydrogel Obturation Materials Comprising PEG Diacrylate or PEG Trimethacrylate.

| Components | | | | | | | |
|---|---|---|---|---|---|---|---|
| (grams) | Ex. 1 | Ex. 2 | Ex. 3 | Ex. 4 | Ex. 5 | Ex. 6 | Ex. 7 |
| Water | 8.8 | 16.5 | 16.8 | 31.0 | 39.0 | 40.0 | 47.6 |
| Poly(ethylene | 60.0 | 52.0 | 48.5 | 23.0 | 24.1 | 22.1 | |
| glycol) diacrylate | | | | | | | |
| Mn 700 (PEGDA) | | | | | | | |
| 8 -arm PEG | | | | 1.0 | 1.0 | 1.0 | |
| 3-arm PEG | | | | | | | 49.1 |
| trimethacrylate | | | | | | | |
| Potassium | | 0.3 | 1.5 | 1.5 | 1.5 | 1.5 | 1.4 |
| persulfate | | | | | | | |
| 1-phenyl-1,2- | 0.6 | 0.6 | 0.6 | 0.6 | 0.6 | 0.6 | |
| propanedione | | | | | | | |
| (PPD) | | | | | | | |
| Triethanolamine | | 0.6 | 0.6 | 0.6 | 0.6 | 0.6 | 1.9 |
| (TEOA) | | | | | | | |
| Dimethylaminoethyl | 0.6 | | | | | | |
| Acrylate | | | | | | | |
| (DMAEA) | | | | | | | |
| [2-(acryloyloxy)- | | | 2.0 | 0.5 | 0.5 | 0.5 | |
| ethyl]-trimethyl- | | | | | | | |
| ammonium | | | | | | | |
| chloride (EGAA) | | | | | | | |
| Calcium | | | | 2.8 | 2.3 | 2.9 | |
| Hydroxide | | | | | | | |
| 5-acrylamido-2,4,6 | 30.0 | 30.0 | 30.0 | 40.0 | 30.0 | 30.0 | |
| triiodo isophthalic | | | | | | | |
| acid | | | | | | | |
| TOTAL | 100.0 | 100.0 | 100.0 | 101.0 | 99.6 | 99.2 | 100.0 |
| Physical | clear, | clear, | clear and | clear and | clear and | clear and | cloudy |
| Appearance | yellow | yellow | yellow | yellow | yellow | yellow | |
| Light cure | 10 s | 10 s | 5 s | 5 s | 5 s | 5 s | |
| Properties | exposure: | exposure: | exposure: | exposure: | exposure: | exposure: | |
| Hardness: | Shore A 77 | Shore A 74.5 | Shore A 85 | Shore A 45 | Shore A 29 | Shore OO 15 | |
| Shore A or OO | DOC: 23 mm | DOC: 19 mm | DOC: 26 mm | DOC: 24 mm | DOC: 25 mm | DOC: 14 mm | |
| DOC: Depth of | 20 s | | 10 s | 10 s | 10 s | 10 s | |
| Cure | exposure: | | exposure: | exposure: | exposure: | exposure: | |
| | Shore A 82 | | Shore A 82 | Shore A 47 | Shore A 41 A | Shore (OO) 52 | |
| | DOC: 27 mm | | DOC: 33 mm | DOC: 26 mm | DOC: 29 mm | DOC: 14 mm | |
| | | | | 20 s | 20 s | 20 s | |
| | | | | exposure: | exposure: | exposure: | |
| | | | | Shore A 65 | Shore A 53 | Shore (OO) 73 | |
| | | | | DOC: 27 mm | DOC: 30 mm | DOC: 15 mm | |
| Work Time | | 1 h 30 m | 30 m | 30 m | 30 m | 15 m | |
| (hours/minutes) | | | | | | | |
| Chemical Cure @ | | Shore A 81 | Shore A 81 | Shore A 42 | Shore A 38.5 | Shore A 45 | Shore A 13 |
| 37° C. for 24 hr | | | | | | | |
| Light exposure | | Shore A 83.5 | Shore A 83 | Shore A 42 | Shore A 39 | Shore A 51 | |
| (10 s); Heat | | | | | | | |
| exposure @ 37° C. | | | | | | | |
| for 24 hours | | | | | | | |
| Notes | | | | pH = 3.16 | pH = 3.37 | pH = 7.20 | stable in |
| | | | | | | | water, |
| | | | | | | | NaOH |

Hydrogels of Examples 1 through 7 were prepared from solutions having between about 8 wt % and 48 wt % water, between about 22 wt % and 60 wt % poly(ethylene glycol) diacrylate and from about 30 wt % to 40 wt % 5-acrylamido-2,4,6 triiodo isophthalic acid as a radiopaque agent. Solutions of Examples 1-3 comprised about 48 wt % to about 60 wt % PEG diacrylate (PEGDA, Mn 700), and Examples 4-6 comprised 31 wt % to 40 wt % PEG diacrylate (PEGDA, Mn 700) combined with about 1 wt % of an 8-arm PEG crosslinking agent. Example 7 comprised about 49 wt % 3-arm PEG trimethacrylate (Sigma-Aldrich, Milwaukee, WI, #416177, trimethylolpropane core) and about 48 wt % water.

The solutions of Example 1 resulted in hydrogels with Shore A hardness values of 77 and 82 after light exposures of 10 seconds and 20 second, respectively. The solutions of Examples 2-6 were both exposed to both light and heat, and Example 7 was exposed to heat. Examples 2 and 3 each comprising about 16 wt % to 17 wt % water were light cured with Example 2 having a measured Shore A hardness of about 74 with an exposure of 10 seconds, and Example 3 which further comprised the quaternary ammonium salt EGAA, had a measured Shore A of about 85 after 5 seconds exposure. Examples 2 and 3 were heated to 37° C. and had a Shore A of 81, and after light curing for 10 seconds and conditioning at 37° C. for 24 hours, each had a measured Shore A hardness value of about 83.

Examples 4-6 each comprising about 30 wt % to 40 wt % water, 1 wt % of 8-arm PEG as a crosslinker, a quaternary ammonium salt, the radiopaque material and calcium hydroxide for solubility, were cured to a Shore A hardness between about 39 and 51, by a combination of exposure to light and heat. Shore A hardness values for hydrogel gels made from the solutions of Examples 4-6 measured about 38 to about 45 after heating at 37° C. overnight. Hydrogels made from solutions comprising PEG methacrylate had a Shore A hardness measurement of about 13 after heating to 37° C. The hydrogels formed herein were suitable for use as an obturation material.

### Examples 8-12

Hydrogels were formed from aqueous poly(ethylene glycol) diacrylate solutions prepared as 2 part systems. The solutions further comprised a water soluble radiopaque material.

For each Example, Part A and Part B solutions were prepared comprising the components provided in Table 2. Final aqueous solutions were combined by mixing Part A and Part B in the ratios provided. Solutions of Examples 8-10 comprised calcium hydroxide; solutions of Examples 11 and 12 comprised sodium hydroxide and were tested for stability and/or solubility.

TABLE 2
Hydrogels Formed From 2-Part Diacrylate Solutions.

| | Ex. 8 | Ex. 9 | Ex. 10 | Ex. 11 | Ex. 12 |
|---|---|---|---|---|---|
| Part A | | | | | |
| Distilled Water | 32.80 | 32.80 | 26.55 | 36.50 | 42.00 |
| Poly(ethylene glycol) | 24.12 | 24.12 | 30.97 | 29.50 | 35.00 |
| diacrylate Mn 700 (PEGDA) | | | | | |
| 1-phenyl-1,2-propanedione | 0.75 | 0.75 | 0.60 | 0.75 | 0.75 |
| (PPD) | | | | | |
| Triethanolamine | 0.75 | 0.75 | 0.60 | 0.75 | 0.75 |
| Sodium Hydroxide | | | | 2.68 | 2.30 |
| Calcium Hydroxide | 3.64 | 3.84 | 4.26 | — | — |
| 4-Methoxyphenol | 0.015 | 0.007 | | | |
| 5-acrylamido-2,4,6 | 37.30 | 37.30 | 39.82 | 30.00 | 20.00 |
| triiodo isophthalic acid | | | | | |
| TOTAL | 99.38 | 99.57 | 102.80 | 100.18 | 100.80 |
| Part B | | | | | |
| Distilled Water | 95.00 | 95.00 | 95.00 | 95.00 | 95.00 |
| Potassium Persulfate | 5.00 | 5.00 | 5.00 | 5.00 | 5.00 |
| TOTAL | 100.00 | 100.00 | 100.00 | 100.00 | 100.00 |
| Mixing Ratio | 5:1 | 5:1 | 1 1 | 1:1 | 1:1 |
| Part A to Part B | by weight | by weight | by volume | by volume | by volume |
| Physical Appearance | part A: clear | part A: clear | part A: clear | clear and | clear and |
| | and brownish | and brownish | and brownish | yellow. No | yellow. No |
| | yellow; part | yellow; part | yellow; part | precipitates | precipitates |
| | B: colorless | B: colorless | B: colorless | observed after | observed after |
| | and clear | and clear | and clear | 5 days | 5 days |
| Radiopacity | 5.3 mmAl | | 2.0 mmAl | | |
| (ratio of Part A to | (5:1) | | (1:1) | | |
| Part B by volume) | | | 3.15 mmAl | | |
| | | | (1.5:1) | | |
| Shore Hardness | 10 s:24 A | 10 s:15 A | 10 s:68 | 10 s:90 | 10 s:68 |
| Light exposure | | | (OO) | (OO)/38 A | (OO)/12 A |
| Work time | 10 m | 10 m | 5 m | 15 seconds | <15-20 seconds |
| Hardness | Shore A | Shore A | | conditioned | conditioned |
| | 24.5 | 14.5 | | 24 h/37 C.: | 24 h/37 C.: |
| | | | | 70 (OO)/15 A | Shore OO |
| | | | | | 85/Shore A 26 |
| Hardness | Shore A | Shore A | | Shore OO | Shore OO |
| [Light Cure 10 s; aging | 22.5 | 15 | | 90/Shore A 32 | 85/Shore A 27 |
| at 37° C. for 24 h] | | | | | |
| Stability of cured | Sample | Sample | | broke into | broke into |
| material in water | stays intact - | stays intact - | | small pieces | small pieces |
| (5 days) | no visible | no visible | | within 5 mins. | within 5 mins. |
| | degradation | degradation | | Solid at 5 days | Solid at 5 days |
| Stability of cured | broke apart | broke apart | completely | | |
| material in LiOH | into small | into small | dissolved | | |
| (pH 12) | pieces | pieces | | | |
| pH of Part A | pH: 7.70 | pH: 7.33 | pH: 7.0 | | |
| Stability: Day 6 | pH = 6.5, | pH = 6.3, | | | |
| at room temp.; | Chem cure at | Chem cure at | | | |
| Shore A Hardness | 37 C. only: 21; | 37 C. only: 19; | | | |
| | Light cure 10 | Light cure 10 | | | |
| | s + Chem cure | s + Chem cure | | | |
| | at 37 C.: 21.5 | at 37 C.: 20.5 | | | |

Hydrogels prepared from the final aqueous solutions comprising 20 wt % or more of a water soluble radiopaque material and a base had a radiopacity of 2 mmAl or higher (e.g., Examples 8 and 10). All solutions formed hard hydrogels after a 5 second exposure with light curing. The hydrogel of Example 10 dissolved after soaking in a lithium hydroxide solution at pH 12.

### Example 13

A radiopaque hydrogel was prepared from an aqueous solution comprising poly(ethylene glycol) diacrylate, a water soluble radiopaque material and further comprised lithium hydroxide prepared as a 2 part aqueous system, with properties as described in Table 3.

Part A and Part B solutions were prepared as above for Examples 1-12, except Parts A and B were combined in a 1.5 (Part A):1 (Part B) by weight ratio. The final composition comprised approximately 55.8 wt % water, approximately 18.5 wt % poly(ethylene glycol) diacrylate Mn 700 (PEGDA), approximately 23.4 wt % 5-acrylamido-2,4,6-triiodo isophthalic acid, approximately 0.3 wt % 1-phenyl-1,2-propanedione (PPD), 0.4 wt % potassium persulfate, approximately 0.3 wt % triethanol amine (TEOA) and approximately 1.3 wt % lithium hydroxide.

TABLE 3
Hydrogel Formed From 2-Part Diacrylate Solution.

| | |
|---|---|
| Shore A Hardness: light exposure 10 seconds | 21 |
| Shore A Hardness: heated to 37° C. for 24 hr | 20 |
| Shore A Hardness: light exposure 10 | 22 |
| seconds and heated to 37° C. for 24 hr | |
| Radiopacity, mmAl | 3.15 |
| Set time | 5 minutes |

### Examples 14 and 15

Hydrogels were formed from aqueous solutions comprising a polyether diamine and a water-soluble radiopaque material.

O,O′-bis(2-aminopropyl) polypropylene glycol-block-polyethylene glycol-block-polypropylene glycol substantially according to Formula (IV) (Jeffamine® ED-2003, Sigma-Aldrich) was combined with components as provided in Table 4. The solution of Example 14 had more than 86 wt % aqueous carrier; Example 15 had about 48% aqueous carrier.

TABLE 4
Hydrogel Formed From Polyether Diamine Solution.

Component

| (grams) | Ex. 14 | Ex. 15 | |
|---|---|---|---|
| Deionized Water | 86.60 | 48.40 | |
| Jeffamine ® ED-2003 diacrylate | 13.40 | 13.70 | |
| 1-phenyl-1,2-propanedione | | 0.60 | |
| (PPD) | | | |
| Triethanolamine (TEOA) | 0.30 | 0.60 | |
| Sodium Hydroxide | | 4.38 | |
| 5-acrylamido-2,4,6 triiodo | | 31.90 | |
| isophthalic acid | | | |
| TOTAL | 100.30 | 99.58 | |
| Physical Appearance | clear and yellow | red solution after | |
| | | a few hours | |
| Set time with light cure | | 5 | seconds (s) |
| Light cure (5 sec) | | Shore OO = 0 | |
| Work Time | 2 minutes (m) | 15 | s |
| Shore Hardness | — | 5.5 | A |
| Aged at @ 37° C. for 24 h | | 74 | (OO) |
| Shore Hardness | — | 5 | A |

| | | |
|---|---|---|
| Light cure (10 s); | | |
| and stored to @ 37° C., | | |
| for 24 hours | | |
| Stability in water | stable | broke into |
| | | small pieces |
| Stability in NaOH | stable | broke into |
| solution (pH 12) | | small pieces |

Solutions comprising 13.4 wt % and 13.7 wt % Jeffamine® ED-2003 diacrylate monomer (Huntsman, Sigma-Aldrich) were mixed with components according to Table 4.

### Example 16

Hydrogels were formed from an aqueous solution comprising poly(ethylene glycol) diacrylamide and a water-soluble radiopaque material.

A solution was prepared by mixing approximately 48.5 g of water, 20 g poly(ethylene glycol) diacrylamide (Mn approximately 1000), 30 g 5-acrylamido-2,4,6 triiodo isophthalic acid, 0.5 g of potassium persulfate, 0.5 g triethanol amine, and 0.5 g 1-phenyl-1,2-propanedione. The samples were exposed to light until hard, then heated to 37° C. for 24 hours.

### Examples 17-29

Curable obturation materials were made that comprised a diacrylate monomer and calcium silicate, each prepared from a three-part system, deliverable to a patient through a hand-held device.

Three-part systems were made according to Tables 5-8 below. For each example, Part A was an aqueous solution comprising the monomer poly(ethylene glycol) diacrylate (PEGDA, Mn 700), 5-acrylamido-2,4,6 triiodo isophthalic acid as a radiopaque agent, triethanolamine co-initiator and calcium hydroxide (Ca(OH)₂) base, in deionized water. Optionally, Part A may further comprises a crosslinker, such as N,N′-methylenebis-acrylamide (MBAA) or 8-arm PEG acrylate.

Part B comprised tricalcium silicate (C3S) (2.7 μm median particle size; Sukgyung AT Co. Ltd., Korea), and either propylene glycol (PG) or diacrylate monomer (PEGDA Mn 700), and optionally, a crosslinker. Part C comprised 5 wt % potassium persulfate (KPS) in deionized water. Components of each part were mixed by stirring to form liquid parts A, B and C. Part A was generally clear and brownish red for Examples 17-29, Part B was white and opaque, and part C was generally colorless and clear. Obturation materials were formed by mixing parts A, B and C in weight ratios with approximate ranges from 1 to 3 parts A: 1 to 3 parts B: 0.2 to 0.5 parts C, as specified in Tables 5-8.

Obturation material properties such as work times, radiopacity, hardness, durability and swelling after soaking in water and lithium hydroxide (LiOH) at approximately pH 12, were observed or measured and recorded in the Tables below.

### Examples 17 and 18

Obturation materials of Example 17 were prepared in five different ratios of parts A:B:C. Obturation materials of Example 18, prepared in three different ratios of parts A:B:C, were substantially similar to Ex. 17 except that propylene glycol in Part B in Ex, 17 was substituted for PEGDA (Mn 700).

TABLE 5
Three-Part Obturation Materials Comprising Water Soluble
Diacrylate-Based Monomer and Tricalcium Silicate.

| Component | | | | | | | | |
|---|---|---|---|---|---|---|---|---|
| (grams) | Example 17 | | | | | Example 18 | | |
| PART A | | | | | | | | |
| H₂0 | 27.25 | | | | | 27.25 | | |
| PEGDA | 27.25 | | | | | 27.25 | | |
| Mn 700 | | | | | | | | |
| Triethanolamine | 1.36 | | | | | 1.36 | | |
| Ca(OH)₂ | 3.50 | | | | | 3.50 | | |
| Radiopaque | 40.87 | | | | | 40.87 | | |
| agent | | | | | | | | |
| TOTAL | 100.23 | | | | | 100.23 | | |
| PART B | | | | | | | | |
| C₃S | 50.00 | | | | | 50.00 | | |
| PG | 50.00 | | | | | | | |
| PEGDA | | | | | | 50.00 | | |
| Mn700 | | | | | | | | |
| TOTAL | 100.00 | | | | | 100.00 | | |
| PART C | | | | | | | | |
| H₂O | 95.00 | | | | | 95.00 | | |
| KPS | 5.00 | | | | | 5.00 | | |
| TOTAL | 100.00 | | | | | 100.00 | | |
| Mix Ratio | | | | | | | | |
| By wt | | | | | | | | |
| Pt A | 1 | 2 | 1 | 3 | 1 | 1 | 2 | 1 |
| Pt B | 1 | 1 | 2 | 1 | 3 | 1 | 1 | 2 |
| Pt C | 0.2 | 0.4 | 0.4 | 0.5 | 0.5 | 0.2 | 0.4 | 0.4 |
| Radiography | | 4.3 | | | | | | |
| (mm Al) | | | | | | | | |
| Work time | 2 mins | 30 sec | 2 mins | 30 sec | 30 sec | 2 mins | 30 sec | 2 mins |
| Soaked in | | Swelling; | | | | | swelling | |
| water | | dissolved | | | | | | |
| | | after 1 wk | | | | | | |
| Soaked in | | Swelling; | | | | | Significant | |
| LiOH pH 12 | | dissolved | | | | | swelling | |
| | | after 1 wk | | | | | | |
| Cured | liquid | | liquid | | liquid | | | liquid |
| material | on top | | on top | | on top | | | on top |
| stored in | after | | after | | after | | | after |
| humidor | 24 h | | 24 h | | 24 h | | | 24 h |
| 37° C. | | | | | | | | |

In Ex. 17, Part B showed separation within about 24 hours, and in Ex. 18 Part B remained homogeneous after about one-week. After mixing parts A, B and C work time for the obturation materials was between 30 seconds and 2 minutes. The resulting cured obturation materials had a radiopacity of 4.3 mmAl. Some swelling of cured obturation materials was observed, and some cured samples of Ex. 17 dissolved after soaking for 1 week at high pH.

### Examples 19-21

Examples 19-21 were prepared similarly to Examples 19 and 20, except they further comprised a multi-arm PEG acrylate. From 1 wt % to 10 wt % 8-arm PEG acrylate [8-Arm(TP) poly(ethylene glycol) acrylate with tripentaerythritol (TP) core, 10 k; JenKem Technology USA] was added to Part A.

TABLE 6
Three-Part Obturation Materials Comprising Water Soluble Diacrylate-
Based Monomer, 8-arm PEG and Tricalcium Silicate.

| Component | | | | | | | | | |
|---|---|---|---|---|---|---|---|---|---|
| (grams) | Example 19 | | | Example 20 | | | Example 21 | | |
| PART A | | | | | | | | | |
| H₂0 | 27.25 | | | 27.25 | | | 27.25 | | |
| PEGDA | 27.25 | | | 27.25 | | | 18.25 | | |
| Mn 700 | | | | | | | | | |
| 8 - arm PEG | 1.00 | | | 1.00 | | | 10.00 | | |
| Triethanolamine | 1.36 | | | 1.36 | | | 1.36 | | |
| Ca(OH)₂ | 3.89 | | | 3.89 | | | 3.97 | | |
| Radiopaque | 40.87 | | | 40.87 | | | 40.87 | | |
| agent | | | | | | | | | |
| TOTAL | 101.62 | | | 101.62 | | | 101.70 | | |
| PART B | | | | | | | | | |
| C₃S | 50.00 | | | 50.00 | | | 50.00 | | |
| PG | 50.00 | | | | | | | | |
| PEGDA | | | | 50.00 | | | 50.00 | | |
| Mn700 | | | | | | | | | |
| TOTAL | 100.00 | | | 100.00 | | | 100.00 | | |
| PART C | | | | | | | | | |
| H₂O | 95.00 | | | 95.00 | | | 95.00 | | |
| KPS | 5.00 | | | 5.00 | | | 5.00 | | |
| TOTAL | 100.00 | | | 100.00 | | | 100.00 | | |
| Mix Ratio | | | | | | | | | |
| By wt | | | | | | | | | |
| Pt A | 1 | 2 | 1 | 1 | 2 | 1 | 1 | 2 | 1 |
| Pt B | 1 | 1 | 2 | 1 | 1 | 2 | 1 | 1 | 2 |
| Pt C | 0.2 | 0.4 | 0.4 | 0.2 | 0.4 | 0.4 | 0.2 | 0.4 | 0.4 |
| Work time | 2 mins | 2 mins | 2 mins | 2 mins | 2 mins | 2 mins | 2 mins | 2 mins | 30 sec |
| Shore | | | | | | | 92 (OO) | 93 (OO) | 91 (OO) |
| Hardness | | | | | | | 74 A | 73 A | 76 A |
| Heat Cure | | | | | | | | | |
| 37° C. | | | | | | | | | |
| Soaked in | dissolved, | | | swelling, | | | swelling, | | |
| water | pH = 12 | | | no cracking | | | no cracking | | |
| Soaked in | expanded | | | swelling, | | | swelling, | | |
| LiOH pH 12 | | | | no cracking | | | no cracking | | |
| Cured | | liquid | liquid | | | | | | |
| material | | after | on top | | | | | | |
| stored in | | 24 h | after | | | | | | |
| humidor | | | 24 h | | | | | | |
| 37° C. | | | | | | | | | |

Part A of Examples 1961 and 20 each comprised 1 wt % 8-arm PEG, and Ex. 21 comprised 10 wt % 8-arm PEG. Ex. 19 comprising approximately 50 wt % PG in Part B, dissolved when soaked in water. Exs. 20 and 21 comprising approximately 50 wt % PEGDA in Part B, showed swelling and no cracking. In Ex. 21 Shore A hardness values increased from 73 to 76 with increasing amounts of part B (tricalcium silicate) in the final obturation material.

### Examples 22-27

Examples 22-27 were prepared substantially similarly to Exs. 17 and 18 and further included MBAA as a crosslinker in either Part A or Part B, as set forth in Table 7. Approximately 1 wt % to 2 wt % MBAA was added to Part A for Exs. 22-27; approximately 1.2 wt % to 3.4 wt % was added to Part B for Exs. 26 and 27.

TABLE 7
Three-Part Obturation Materials Comprising Water Soluble Diacrylate-Based Monomer, MBAA and Tricalcium Silicate.

| Component | | | | | | | | | | |
|---|---|---|---|---|---|---|---|---|---|---|
| (grams) | Example 22 | | | Example 23 | | | Example 24 | Example 25 | Example 26 | Example 27 |
| PART A | | | | | | | | | | |
| H₂0 | 27.25 | | | 27.25 | | | 27.25 | 27.25 | 27.25 | 27.25 |
| PEGDA | 27.25 | | | 27.25 | | | 27.25 | 27.25 | 27.25 | 27.25 |
| Mn 700 | | | | | | | | | | |
| MBAA | 1.00 | | | 1.00 | | | 1.72 | 2.06 | | |
| Triethanolamine | 1.36 | | | 1.36 | | | 1.36 | 1.36 | 0.90 | 0.90 |
| Ca(OH)₂ | 3.89 | | | 3.89 | | | 3.89 | 3.89 | 3.89 | 3.89 |
| Radiopaque | 40.87 | | | 40.87 | | | 40.87 | 40.87 | 40.87 | 40.87 |
| agent | | | | | | | | | | |
| TOTAL | 101.62 | | | 101.62 | | | 102.34 | 102.68 | 100.16 | 100.16 |
| PART B | | | | | | | | | | |
| C₃S | 50.00 | | | 50.00 | | | 50.00 | 50.00 | 50.00 | 48.31 |
| PG | | | | 50.00 | | | | | | |
| PEGDA | 50.00 | | | | | | 50.00 | 50.00 | 50.00 | 48.31 |
| Mn 700 | | | | | | | | | | |
| MBAA | | | | | | | | | 1.20 | 3.38 |
| TOTAL | 100.00 | | | 100.00 | | | 100.00 | 100.00 | 101.20 | 100.00 |
| PART C | | | | | | | | | | |
| H₂O | 95.00 | | | 95.00 | | | 95.00 | 95.00 | 95.00 | 95.00 |
| KPS | 5.00 | | | 5.00 | | | 5.00 | 5.00 | 5.00 | 5.00 |
| TOTAL | 100.00 | | | 100.00 | | | 100.00 | 100.00 | 100.00 | 100.00 |
| Mixing | | | | | | | | | | |
| ratio by | | | | | | | | | | |
| wt | | | | | | | | | | |
| Pt A | 1 | 2 | 1 | 1 | 2 | 1 | 2 | 2 | 2 | 2 |
| Pt B | 1 | 1 | 2 | 1 | 1 | 2 | 1 | 1 | 1 | 1 |
| Pt C | 0.2 | 0.4 | 0.4 | 0.2 | 0.4 | 0.4 | 0.4 | 0.4 | 0.4 | 0.4 |
| Work | 2 mins | 2 mins | 2 mins | 2 mins | 2 mins | 2 mins | 30 sec | 30 sec | 30 sec | 30 sec |
| time | | | | | | | | | | |

| | | | | |
|---|---|---|---|---|
| Shore | 92 (OO) | 93 (OO) | 95(OO) | 95 (OO) |
| Hardness | 76 A | 75 A | 70 A | 78 A |
| Heat | | | | |
| Cure | | | | |
| 37° C. | | | | |

| Cured gel | dissolved | dissolved | 24% | 16% | swelling |
|---|---|---|---|---|---|
| soaked in | | | swelling | swelling | |
| water | | | | | |
| Cured gel | dissolved | dissolved | swelling, cracking | | swelling |
| soaked in | | | | | |
| LiOH pH 12 | | | | | |

| | | | | | | | | |
|---|---|---|---|---|---|---|---|---|
| Cured | 37° C., | 37° C., | 37° C., | 37° C., | 37° C., | 37° C., | not | not |
| material | liquid | liquid | liquid | liquid | liquid | liquid | completely | completely |
| stored in | on top | on top | on top | after | after | after | dissolved | dissolved |
| humidor | after | after | after | 4 days | 4 days | 4 days | in part B | in part B |
| @ 37 C. | 24 h | 24 h | 24 h | | | | | |

Cured obturation materials of Exs. 22 and 23, having about 1 wt % MBAA in Part A, dissolved after soaking in water and lithium hydroxide (pH 12). For cured obturation materials of Exs. 24 and 25, having (approximately 1.7 wt % and 2 wt %, MBAA respectively) higher levels of MBAA than Exs. 22 and 23 in Part A, swelling and cracking was observed but materials did not dissolve. For cured obturation materials of Exs. 26 and 27, having 1.2 wt % and 3.4 wt % MBAA in Part B, respectively, no cracking was observed. Cured obturation materials of Exs. 24 and 25 had Shore A hardness values of 76 and 75, respectively, while cured obturation materials of Exs. 26 and 27 had Shore A values of 70 and 78, respectively.

### Examples 28 and 29

Examples 28 and 29 were made substantially according to the Example 24, with modifications according to Table 8. To test the reactivity of tricalcium silicate, the amounts of tricalcium silicate were varied in Exs. 28 and 29. The three-part systems further comprised MBAA crosslinker in Part A, and PEGDA in Part B.

TABLE 8
Three-Part Obturation Materials Comprising Water Soluble
Diacrylate-Based Monomer, MBAA and Tricalcium Silicate.

Component

| (grams) | Ex. 28 | | Ex. 29 | |
|---|---|---|---|---|
| Part A | | | | |
| H2O | 27.25 | | 27.25 | |
| PEGDA Mn 700 | 27.25 | | 27.25 | |
| MBAA | 1.75 | | 1.75 | |
| Triethanolamine | 0.90 | | 0.90 | |
| Calcium Hydroxide | 4.07 | | 4.07 | |
| Radiopaque agent | 40.87 | | 40.87 | |
| TOTAL | 102.09 | | 102.09 | |
| Part B | | | | |
| C₃S | 25.00 | | 75.00 | |
| PEGDA Mn 700 | 75.00 | | 25.00 | |
| TOTAL | 100.00 | | 100.00 | |
| Part C | | | | |
| H2O | 95.00 | | 95.00 | |
| KPS | 5.00 | | 5.00 | |
| TOTAL | 100.00 | | 100.00 | |
| Mixing ratio by weight | | | | |
| Pt A | 2 | | 2 | |
| Pt B | 1 | | 1 | |
| Pt C | 0.4 | | 0.4 | |
| Work time | 30 | sec | 30 | sec |
| Shore Hardness | 91 | (OO) | 90 | (OO) |
| Heat Cure: 37° C. | 68 | A | 64 | A |

| | | |
|---|---|---|
| Cured gel soaked in water | 23% swelling, | 23% swelling |
| | no cracking | |
| Cured gel soaked in | swelling, cracking | swelling, cracking |
| LiOH pH 12 | | |

### Examples 30-38

Hydrogels were formed from aqueous solutions comprising diacrylate, diacrylamide or a 3-arm PEG triacrylamide, and a water-soluble radiopaque material.

The solutions further comprised a quaternary ammonium salt to impart antimicrobial activity, and an ionic (5-acrylamido-2,4,6 triiodo isophthalic acid) or non-ionic iodinated acrylate monomer as an X-ray contrast agent. Solutions were exposed to light until hard, then heated to 37° C. for 24 hours.

TABLE 9
Hydrogels Prepared From Water Soluble Acrylate-Based and Acrylamide-Based Monomers.

| Component | | | | | | | | | |
|---|---|---|---|---|---|---|---|---|---|
| (grams) | Ex. 30 | Ex. 31 | Ex. 32 | Ex. 33 | Ex. 34 | Ex. 35 | Ex. 36 | Ex. 37 | Ex. 38 |
| Distilled Water | 48.4 | 48.4 | 48.4 | 48.4 | 48.4 | 48.4 | 48.4 | 48.4 | 48.4 |
| Poly(ethylene glycol) | 20 | 20 | 20 | | | | | | |
| diacrylate Mn | | | | | | | | | |
| approx. 700 | | | | | | | | | |
| (PEGDA) | | | | | | | | | |
| PEG diacrylamide | | | | 20 | 20 | 20 | | | |
| (Mn approx. 1000) | | | | | | | | | |
| 3-Arm PEG | | | | | | | 20 | 20 | 20 |
| Triacrylamide (Mn | | | | | | | | | |
| approx. 1000) | | | | | | | | | |
| Potassium Persulfate | 0.5 | 0.5 | 0.5 | 0.5 | 0.5 | 0.5 | 0.5 | 0.5 | 0.5 |
| 1-phenyl-1,2- | 0.5 | 0.5 | 0.5 | 0.5 | 0.5 | 0.5 | 0.5 | 0.5 | 0.5 |
| propanedione (PPD) | | | | | | | | | |
| Triethanolamine | 0.5 | 0.5 | 0.5 | 0.5 | 0.5 | 0.5 | 0.5 | 0.5 | 0.5 |
| (TEOA) | | | | | | | | | |
| [2-(acryloyloxy)- | 0.1 | 0.1 | 0.1 | 0.1 | 0.1 | 0.1 | 0.1 | 0.1 | 0.1 |
| ethyl]-trimethyl- | | | | | | | | | |
| ammonium chloride | | | | | | | | | |
| (EGAA) | | | | | | | | | |
| 5-acrylamido-2,4,6 | 30 | | | 30 | | | | | |
| triiodo isophthalic | | | | | | | | | |
| acid | | | | | | | | | |
| 5-acryloylamino- | | 30 | | | 30 | | | | |
| N,N′-bis-[(2- | | | | | | | | | |
| hydroxy-1,1-bis- | | | | | | | | | |
| hydroxymethyl- | | | | | | | | | |
| ethylcarbamoyl)- | | | | | | | | | |
| methyl]-2,4,6- | | | | | | | | | |
| triiodoisophthalamide | | | | | | | | | |
| 5-acryloylamino-N- | | | 30 | | | 30 | | | |
| (2,3- | | | | | | | | | |
| dihydroxypropyl)-N′- | | | | | | | | | |
| (2-hydroxy-1,1-bis- | | | | | | | | | |
| hydroxymethylethyl)- | | | | | | | | | |
| 2,4,6- | | | | | | | | | |
| triiodoisophthalamide | | | | | | | | | |
| TOTAL | 100 | 100 | 100 | 100 | 100 | 100 | 100 | 100 | 100 |

### Examples 39-47

Hydrogels were formed from two-part aqueous solutions comprising Jeffamine diacrylate or a triacrylamide, and 2,4,6-Triiodo-5-[(1-oxo-2-propen-1-yl)amino]-1,3-benzenedicarboxylic acid or 5-acryloylamino-N-(2,3-dihydroxypropyl)-N′-(2-hydroxy-1,1-bis-hydroxymethylethyl)-2,4,6-triiodoisophthalamide as radiopaque materials.

TABLE 10A
Two-Part Aqueous Obturation Materials

| Components (g) | Ex. 39 | Ex. 40 | Ex. 41 | Ex. 42 | Ex. 43 |
|---|---|---|---|---|---|
| Part A | | | | | |
| Distilled H2O | 27.92 | 33.06 | | 15 | 15 |
| Triacrylamide | 36.38 | | 33.4 | 28.95 | 28.95 |
| | | | (20% H2O) | (20% H2O) | (20% H2O) |
| Jeffamine Diacrylamide 900 | | 27.08 | | | |
| 2,4,6-Triiodo-5-[(1-oxo-2- | | 35.15 | | 36.23 | 36.23 |
| propen-1-yl)amino]-1,3- | | | | | |
| benzenedicarboxylic acid | | | | | |
| Triethanolamine | 0.7 | 0.6 | 0.6 | 0.6 | 0.6 |
| Sodium Hydroxide | | 4.11 | | 4.22 | 4.22 |
| 5-acryloylamino-N- | 35 | | 45 | | |
| (2,3-dihydroxypropyl)-N′- | | | | | |
| (2-hydroxy-1,1-bis- | | | | | |
| hydroxymethylethyl)- | | | | | |
| 2,4,6-triiodoisophthalamide | | | | | |
| Propolene Glycol | | | 21 | 15 | 15 |
| Part B | | | | | |
| Distill H2O | 97 | 97 | 97 | 99.25 | 95 |
| KPS | 3 | 3 | 3 | 0.75 | |
| AIPH | | | | | 5 |
| Physical Properties//Part | Part | Part | Part | Part | Part |
| A:Part B Ratio | A(1.5):Part | A(1.5):Part | A(1.5):Part | A(1.5):Part | A(1.5):Part |
| | B (1) | B (1) | B (1) | B (1) | B (1) |
| Work Time (23° C.) | 1′30″ | 6′50″ | 1′30 | 3′ | |
| Set Time (37° C.) | 2′ | 7′30″ | | 4′ | |
| Light Cure 10″ | | 90 (OO), | 64(HA) | 14(HA) | 17 (HA) |
| | | 28 (HA) | | | |
| Heat Cure @37 C. | 85(OO) | | | | 21 (HA) |
| Self Cure @RT | | | | | 57 (HA) |
| Self cure @37 C. | | | | | 57(HA) |
| Condition for 24 hrs | | | | | |
| Light 10 (s) and heat | | | 67 (HA) | 54(HA) | |
| cure @37 C. | | | | | |
| Condition for 24 hrs | | | | | |
| Light 10 (s) and heat | | | | 62(HA) | 63 (HA) |
| cure @37C. | | | | | |
| Condition for 5 days | | | | | |
| Radiopacity | | | 2.96 | | |
| Stability in H2O | Day 10: No | | | | |
| | degradation, | | | | |
| | pH = 3.08 | | | | |
| Stability in NaOH | Day 1: No | | | | |
| solution | degradation, | | | | |
| | sample turned | | | | |
| | brown. | | | | |
| | Day 10: No | | | | |
| | degradation. | | | | |
| Stability Test @37 C. | | | | Day 6: | Day 6: |
| | | | | Homogeneous, | Homogeneous, |
| | | | | WT/ST: 3′/4′, | Heat Cure for |
| | | | | 10″ LC: 35 (HA) | 30′: 27 (HA) |
| Observation/Comments | Homogeneous | Homogeneous. | | — | |

“HA”: Shore A scale hardness
“OO”: Shore OO scale hardness

TABLE 10B
Two-Part Aqueous Obturation Materials

| Component (g) | Ex. 44 | Ex. 45 | Ex. 46 | Ex. 47 |
|---|---|---|---|---|
| Part A | | | | |
| Distilled H2O | 15 | 15 | | 6.83 |
| Triacrylamide | 28.95 | 27.55 | 28.22 | 29.5 |
| | (20% H2O) | (20% H2O) | (20% H2O) | (20% H2O) |
| 2,4,6-Triiodo-5-[(1-oxo-2- | 36.23 | 36.23 | | 39.03 |
| propen-1-yl)amino]-1,3- | | | | |
| benzenedicarboxylic acid | | | | |
| Triethanolamine | 0.6 | 2 | 0.6 | 0.59 |
| Sodium Hydroxide | 4.22 | 4.22 | | 4.53 |
| 5-acryloylamino-N-(2,3- | | | 55 | |
| dihydroxypropyl)-N′-(2- | | | | |
| hydroxy-1,1-bis- | | | | |
| hydroxymethylethyl)- | | | | |
| 2,4,6-triiodoisophthalamide | | | | |
| Propolene Glycol | 15 | 15 | 16.18 | 19.52 |
| Part B | | | | |
| Distill H2O | 97 | 99.25 | 97 | 99.25 |
| KPS | 0.75 | 0.75 | 3 | 0.75 |
| Physical Properties//Part | Part | Part | Part | Part |
| A:Part B Ratio | A(1.5):Part | A(1.5):Part | A(1.5):Part | A(1.5):Part |
| | B (1) | B (1) | B (1) | B (1) |
| Work Time (23° C.) | 3′ | 1′ | 9′30″ | 12′30″ |
| Set Time (37° C.) | 4′ | | | |
| Light Cure 10″ | | 19(HA) | 10″ LC (soft) | 18(HA), 85 |
| | | | after 1 h15 m | (OO) |
| | | | @37° C. (32 HA) | |
| Heat Cure @37 C. | | | after 1 h30 m | |
| | | | @37° C. (44.3 HA) | |
| Radiopacity | | Sample prep: | Initial: 3.95 and | Sample prep: |
| | | 10″ LC each | 4.27 (1 mm mould) | 10″ each spot |
| | | spot and | 3 days in water: | and condition |
| | | condition in | 2.47 and 2.56 | in humidor |
| | | humidor for | Repeated: | for 2 days. |
| | | 2 days. | Sample 10″ | Initial: |
| | | Initial: | each spot and | Sample 1. |
| | | 1. 3.06 (disk) | condition in | Mould: 4.12 |
| | | 2. 3.05 (disk) | humidor for | Sample 2. |
| | | 4 days soaking in | overnight. | Mould: 4.06 |
| | | 1. 2.70 (water) | Initial 4.06 | After 4 days |
| | | 2. 2.73 (PBS) | 2 days in | soaking in |
| | | 1 week soaking in | water: 2.46 | 1. Mould: 4.15 |
| | | 1. 2.56 (water) | Repeated: | 3(water) |
| | | 2. 2.77 (PBS) | Sample 10″ | 2. Mould: 3.54 |
| | | | each spot and | (PBS) |
| | | | condition in | After 1 wk |
| | | | humidor for | soaking in |
| | | | 2 days. | 1. Mould: 3.83 |
| | | | Initial: | (water) |
| | | | 1. 3.86 | 2. Mould: 3.23 |
| | | | 2. 3.59 | (PBS solution) |
| | | | After 4 days | |
| | | | soaking in | |
| | | | 1. 3.12 (water) | |
| | | | 2. 3.10 (PBS) | |
| | | | After 1 wk | |
| | | | soaking in | |
| | | | 1. 3.24 (water) | |
| | | | 2. 3.07 (PBS) | |
| Stability @ RT | 1 wk @RT: 3′/4′ | | | |
| Stability Test @37 C. | 1 wk: 3′/4′ WT/ST | Initial: 1′ | | |
| | 2 wks: 15′ WT | 4 wks @ RT: | | |
| | 3 wks: 20′ WT | 1′00″; 10″ LC: | | |
| | 4 wks: using | 23.6 (HA) | | |
| | 0.75% KPS | 1 wk: 1′10″ | | |
| | solution in Part | WT; 10″ LC: | | |
| | B, light cure | 24(HA) | | |
| | 40″, it is not | 2 wks: 1″30″ | | |
| | curing. | WT; 10″ LC | | |
| | | OK. HA after | | |
| | | 30 minutes | | |
| | | @RT 31(HA) | | |
| | | 3 wks: 3′30″ | | |
| | | WT; 10″ LC | | |
| | | OK. Hardness | | |
| | | @37° C. humidor | | |
| | | condition after | | |
| | | 30 minutes. 30 | | |
| | | (HA) | | |
| | | 4 wks: 4′15″ | | |
| | | WT; 20″ LC, | | |
| | | 21 (HA) | | |
| Stability Test @50 C. | 1 wk: WT: 30′ | 1 wk: 7′ WT; | | |
| | 2 wks: WT: | 10″ LC and | | |
| | longer than 50′, | Heat cure | | |
| | no gel. | @37 C. after | | |
| | 10″ light curing | 2 hours: 30.5 | | |
| | and heat cured | (HA) | | |
| | in oven 37 C. | 2 wks: 9′30″ WT | | |
| | after 2 hr: 95 | 3 wks: 15′ WT; | | |
| | (OO), 27 (HA) | 10″ LC, not | | |
| | 3 wks: WT @ | curing. | | |
| | 1 hour, no gel | Hardness of | | |
| | 10″ light curing | sample @ 37° C. | | |
| | (not cured), | humidor | | |
| | place in oven | condition after | | |
| | 37 C. overnight. | 2 hours. 41.5 | | |
| | 4 wks: using | (HA) | | |
| | 3% KPS | 4 wks: 15′ 40″ | | |
| | solution in part | WT; 40″ light | | |
| | B mix with part | cured: 21 (HA) | | |
| | A. Light cure 1 | | | |
| | minute, not curing. | | | |
| Observation/Comments | Both samples | Both samples | | |
| | @37° C. and | @37° C. and | | |
| | 50° C. are | 50° C. are | | |
| | homogeneous. | homogeneous | | |
| | | after 3 weeks. | | |

“HA”: Shore A scale hardness
“OO”: Shore OO scale hardness

### Examples 48-61

Examples 48-61 were prepared substantially similar to Examples 39-47, aside from modifications according to Tables 11A-11C. Various monomers were evaluated with regard to % swelling. Swelling was determined as follows using a 10 mm diameter×6 mm depth mold:

$$\% Swelling = \frac{( \begin{matrix}diameter after soaking - \\ diameter before soaking\end{matrix} )}{diameter before soaking} ⨯ 100$$

TABLE 11A
Two-Part Aqueous Obturation Materials

| Component | Ex. 48 | Ex. 49 | Ex. 50 | Ex. 51 |
|---|---|---|---|---|
| Part A | | | | |
| Distilled H2O | 30.8 | 30.08 | 14.99 | 15 |
| Poly(ethylene glycol) | 28.9 | | | |
| Diacrylate Mn 700 (PEGDA) | | | | |
| Jeffamine Diacrylamide 900 | | 28.9 | 27.53 | 27.58 |
| | | | (20% H2O) | |
| 5-acrylamido-2,4,6 | 36.23 | 36.23 | 36.2 | 36.15 |
| triiodo isophthalic acid | | | | |
| Triethanolamine | 0.6 | 0.6 | 2.03 | 2 |
| Sodium Hydroxide | 4.19 | 4.19 | 4.25 | 4.27 |
| Propolene Glycol | | | 14.99 | 15 |
| Part B | | | | |
| Distilled H2O | 97 | 97 | 99.25 | 99.25 |
| Potassium Persulfate | 3 | 3 | 0.75 | 0.75 |
| Physical Properties | | | | |
| (mix ratio Part A:Part B = 1.5:1) | | | | |
| Work Time (23° C.) | | | 50″ | 3′30″ |
| | | | | 5′30″ (Part A:Part |
| | | | | B = 2.5:1) |
| Light Cure (10 sec) | | | 22 (HA) | light cured 40″ |
| | | | | 80 (OO) |
| Light 10 sec and heat | | | 34.7 (HA) | 95 (OO) |
| cure @37 C. for 24 h | | | | |
| Radiopacity (mm Al) | 3.85 initial | | 3.01 | 3.61 (mix ratio |
| | 2.34 (1 wk | | | Part A/Part B = |
| | soak in water) | | | 2.5/1) |
| Swelling (24 h in water, %) | 16.4 | 19.4 | | 21.3 |
| Swelling (24 h in PBS, %) | 12.9 | 14.9 | 14.6 | 14.1 |

TABLE 11B
Two-Part Aqueous Obturation Materials

| Component | Ex. 52 | Ex. 53 | Ex. 54 | Ex. 55 | Ex. 56 | Ex. 57 |
|---|---|---|---|---|---|---|
| Part A | | | | | | |
| Distilled H2O | 30.08 | 30.08 | 15 | 6.81 | 14.18 | 15 |
| 3-Arm PEG Triacrylamide (Mn | 28.9 | 33.09 (20% | 27.55 (20% | 29.49 (20% | 31.21 | 27.55 |
| approx. 1000) | | H2O) | H2O) | H2O) | | |
| 5-acrylamido-2,4,6 triiodo | 36.23 | | 36.23 | 38.9 | 34.26 | 36.23 |
| isophthalic acid | | | | | | |
| Triethanolamine | 0.6 | 0.6 | 2 | 2.01 | 2.13 | 2 |
| Sodium Hydroxide | 4.19 | | 4.22 | 4.73 | 4.04 | 4.27 |
| 5-acryloylamino-N-(2,3- | | 36.23 | | | | |
| dihydroxypropyl)-N′-(2-hydroxy- | | | | | | |
| 1,1-bis-hydroxymethylethyl)-2,4,6- | | | | | | |
| triiodoisophthalamide | | | | | | |
| Propolene Glycol | | | 15 | 18.05 | 14.18 | 15 |
| Part B | | | | | | |
| Distilled H2O | 97 | 97 | 99.25 | 97 | 99.25 | 99.25 |
| Potassium Persulfate | 3 | 3 | 0.75 | 3 | 0.75 | 0.75 |
| Physical Properties | | | | | | |
| (mix ratio Part A:Part B = 1.5:1) | | | | | | |
| Work Time (23° C.) | | 1′ | 1′ | 1′ | 2′ | 2′ |
| Light Cure (10 sec) | | | 19 (HA) | 22 (HA) | 75 (OO) | 95 (OO), |
| | | | | | | 30(HA) |
| Light 10 sec and heat | | | | | 95(OO), | 95 (OO), |
| cure @37 C. for 24 h | | | | | 27 (HA) | 39 (HA) |
| Radiopacity (mm Al) | | | Initial: 3.1 | Initial: 3.24 | | 3.4 |
| | | | 4 days | After 1 | | |
| | | | soaking in | soaking in | | |
| | | | 1. 2.70 | PBS: 2.63 | | |
| | | | (water) | | | |
| | | | 2. 2.73 | | | |
| | | | (PBS) | | | |
| | | | 1 week | | | |
| | | | soaking in | | | |
| | | | 1. 2.56 | | | |
| | | | (water) | | | |
| | | | 2. 2.77 | | | |
| | | | (PBS) | | | |
| | | | 6 weeks | | | |
| | | | soaking in | | | |
| | | | 1. 2.61 | | | |
| | | | (water) | | | |
| | | | 2. 2.78 | | | |
| | | | (PBS) | | | |
| Swelling (24 h in water, %) | 12.4 | | | | 16.1 | 16.0 |
| Swelling (24 h in PBS, %) | 6.9 | 15.3 | 8.0 | 11.2 | 11.7 | 12.4 |

TABLE 11C
Two-Part Aqueous Obturation Materials

| Component | Ex. 58 | Ex. 59 | Ex. 60 | Ex. 61 |
|---|---|---|---|---|
| Part A | | | | |
| Distilled H2O | 30.08 | 15 | 15 | 15 |
| 3-Arm PEG Triacrylamide | 14.45 | 18.956 | 21.664 | 24.372 |
| (Mn approx. 1000) | | | | |
| Jeffamine Diacrylamide 900 | 14.45 | 8.124 | 5.416 | 2.708 |
| 5-acrylamido-2,4,6 | 36.23 | 36.23 | 36.23 | 36.23 |
| triiodo isophthalic acid | | | | |
| Triethanolamine | 0.6 | 2 | 2 | 2 |
| Sodium Hydroxide | 4.19 | 4.69 | 4.69 | 4.69 |
| Propolene Glycol | | 15 | 15 | 15 |
| Part B | | | | |
| Distilled H2O | 97 | 99.25 | 99.25 | 99.25 |
| Potassium Persulfate | 3 | 0.75 | 0.75 | 0.75 |
| Physical Properties | | | | |
| (mix ratio Part A:Part B = 1.5:1) | | | | |
| Swelling (24 h in water, %) | 14.4 | 17.6 | 16.3 | 16.0 |
| Swelling (24 h in PBS, %) | 11.4 | 11.0 | 9.9 | 10.5 |

### Examples 62 and 63

Examples 62 and 63 were prepared substantially similar to Example 24, aside from modifications according to Table 12. To increase the radiopacity of the obturation material, ytterbium fluoride as added to Part B of the system. Fumed silica was also added to stabilize the dispersion.

TABLE 12
Paste/Paste Aqueous Obturation Materials with High Radiopacity

| Component | Ex. 62 | Ex. 63 |
|---|---|---|
| Part A | | |
| Distilled water | 9.10 | 9.10 |
| Propylene glycol | 4.14 | 4.14 |
| 5-acrylamido-2,4,6 triiodo isophthalic acid | 28.00 | 28.00 |
| YbF100 | 27.20 | 27.20 |
| Fumes Silica (M5F) | 1.00 | 1.00 |
| Jeffamine Diacrylamide 900 | 25.25 | 25.25 |
| Sodium Hydroxide | 3.31 | 3.31 |
| Triethanolamine | 2.00 | 2.00 |
| Part B | | |
| Distilled water | 37.25 | 10 |
| KPS | 0.75 | 0.75 |
| Propylene glycol | 30 | 50 |
| YbF3 | 30 | 35 |
| Fumes Silica (M5F) | 2 | 4.25 |
| Physical Properties | | |
| Radiopacity (Part A/Part B = 2.5/1) | 6.5 | 6.5 |

While certain embodiments have been described, these embodiments have been presented by way of example only and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products. For example, any of the components for an energy storage system described herein can be provided separately, or integrated together (e.g., packaged together, or attached together) to form an energy storage system.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount, depending on the desired function or desired.

The headings contained in this document, if any, are for convenience only and do not necessarily affect the scope or meaning of the devices and methods disclosed herein.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A curable mixture of ingredients, comprising:
(a) a water-soluble acrylate-based monomer, a water-soluble acrylamide-based monomer, a water-soluble chelating monomer, or a mixture thereof;
(b) a free-radical polymerization initiator;
(c) a radiopaque material, wherein the radiopaque material is selected from the group consisting of 2,4,6-Triiodo-5-[(1-oxo-2-propen-1-yl)amino]-1,3-benzenedicarboxylic acid, 5-acryloylamino-N,N′-bis-[(2-hydroxy-1,1-bis-hydroxymethyl-ethylcarbamoyl)-methyl]-2,4,6-triiodoisophthalamide, 5-acryloylamino-N-(2,3-dihydroxypropyl)-N′-(2-hydroxy-1,1-bis-hydroxymethylethyl)-2,4,6-triiodoisophthalamide, and combinations thereof; and
(d) an aqueous carrier;
wherein the ingredients (a), (b), (c), and (d) are selected to provide the curable mixture with properties suitable for use as a tooth filling after curing the mixture to form a cured mixture by polymerization of ingredient (a) that is initiated by ingredient (b).

2. The curable mixture of claim 1, wherein the free radical initiator comprises a light initiator, thermal initiator, chemical initiator or a combination thereof.

3. The curable mixture of claim 1 or 2, wherein the water-soluble acrylamide-based monomer comprises 3-acrylamidopropyl trimethylammonium chloride, 3-methacrylamidopropyl trimethylammonium chloride, 3-acrylamidopropyl trimethylammonium methyl sulfate, 3-methacrylamidopropyl trimethylammonium methyl sulfate, Jeffamine (ED-900) diacrylate, Jeffamine (ED-2003) diacrylate, poly(ethylene glycol) diacrylamide, 3-Arm poly(ethylene glycol) triacrylamide, or a combination thereof.

4. The curable mixture of any of claims 1 to 3, wherein the water-soluble acrylate-based monomer comprises [2-(methacryloyloxy)ethyl] trimethylammonium chloride, [2-(acryloyloxy)ethyl] trimethylammonium chloride, [2-(acryloyloxy)ethyl] trimethylammonium methyl sulfate, [2-(methacryloyloxy)ethyl] trimethylammonium methyl sulfate, (hydroxyethyl)methacrylate (HEMA), or a combination thereof.

5. The curable mixture of any of claims 1 to 4, wherein the water-soluble acrylate-based monomer comprises poly(ethylene glycol) diacrylate, ethoxylated trimethylolpropane triacrylate, or a combination thereof.

6. The curable mixture of any one of claims 1 to 5, wherein the chelating monomer comprises 4-methacryloxyethyl trimellitic acid (4-MET) or glycerol phosphate dimethacrylate (GPDM).

7. The curable mixture of any one of claims 1 to 6, wherein the radiopaque material comprises 2,4,6-Triiodo-5-[(1-oxo-2-propen-1-yl)amino]-1,3-benzenedicarboxylic acid.

8. The curable mixture of claim 7, wherein the radiopaque material comprises 5-acryloylamino-N,N′-bis-[(2-hydroxy-1,1-bis-hydroxymethyl-ethylcarbamoyl)-methyl]-2,4,6-triiodoisophthalamide.

9. The curable mixture of any one of claims 1 to 6, wherein the radiopaque material comprises 5-acryloylamino-N-(2,3-dihydroxypropyl)-N′-(2-hydroxy-1,1-bis-hydroxymethylethyl)-2,4,6-triiodoisophthalamide.

10. The curable mixture of claim 9, wherein the radiopaque material comprises 5-acryloylamino-N,N′-bis-[(2-hydroxy-1,1-bis-hydroxymethyl-ethylcarbamoyl)-methyl]-2,4,6-triiodoisophthalamide, 2,4,6-Triiodo-5-[(1-oxo-2-propen-1-yl)amino]-1,3-benzenedicarboxylic acid, or a combination thereof.

11. The curable mixture of any one of claims 1 to 10, provided in two parts, each of which comprises a liquid.

12. The curable mixture of claim 11, wherein each of the two liquid part is degassed.

13. The curable mixture of any one of claims 1 to 12, further comprising a polymerization cross-linker.

14. The curable mixture of claim 13, wherein the polymerization cross-linker comprises N, N′-methylenebis(acrylamide) (MBAA), triethylene glycol dimethacrylate (TEGDMA), or a combination thereof.

15. The curable mixture of ingredients of claim 1, wherein the free radical initiator comprises a light initiator.

16. The curable mixture of ingredients of claim 15, comprising camphorquinone (CQ), 7,7-dimethyl-2,3-dioxobicyclo[2.2.1] heptane-1-carboxylic acid (CC Q), 1-phenyl-1,2-propanedione (PPD), 2,2′-azobis[2-methyl-N-(2-hydroxyethyl)propionamide (VA-086).

17. The curable mixture of ingredients of claim 15, comprising a co-initiator selected from N-phenylglycine, 2-pyrrolidinone, dimethylaminoethyl acrylate (DMAEA), triethanolamine (TEOA), 1-vinyl-2-pyrrolidone and L-arginine.

18. The curable mixture of ingredients of any one of claims 1 to 14, wherein the free radical initiator comprises a heat initiator.

19. The curable mixture of ingredients of claim 18, wherein the heat initiator comprises 2,2′-azobis[2-(2-imidazolin-2-yl) propane] dihydrochloride.

20. The curable mixture of ingredients of any one of claims 1 to 14, wherein the initiator comprises potassium persulfate.

21. The curable mixture of ingredients of claim 20, further comprising a co-initiator selected from N-phenylglycine, 2-pyrrolidinone, dimethylaminoethyl acrylate (DMAEA), triethanolamine (TEOA), 1-vinyl-2-pyrrolidone and L-arginine.

22. The curable mixture of ingredients of any one of claims 1 to 21, further comprising methacrylic acid.

23. The curable mixture of ingredients of any one of claims 1 to 22, comprising from wt % to 60 wt % of the aqueous carrier, based on the weight of the curable mixture.

24. The curable mixture of ingredients of any one of claims 1 to 23, wherein the aqueous carrier has a pH in the range of about 7.0 to about 8.4.

25. An obturation material for use as a radiopaque tooth filling after curing, the obturation material, provided as two or more liquid parts, comprising:
(a) a water-soluble acrylate-based monomer, a water-soluble acrylamide-based monomer, a water-soluble chelating monomer, or a mixture thereof, selected from the group consisting of a multi-arm poly(ethylene glycol) acrylate, a multi-arm poly(ethylene glycol) acrylamide, an acrylate functionalized polyether, an acrylamide functionalized polyether, a methacrylamide functionalized polyether, and combinations thereof;
(b) a free-radical polymerization initiator;
(c) a radiopaque material; and
(d) an aqueous carrier;
wherein at least one liquid part has a viscosity less than 100 cP (at 25° C.), wherein (a), (b), (c), and (d) are selected to form a curable mixture, and wherein the obturation material is curable in a tooth by polymerization of ingredient (a) that is initiated by ingredient (b).

26. The obturation material of claim 25, wherein at least one liquid part has a viscosity less than 20 cP (25° C.).

27. The obturation material of claim 25 or 26, wherein at least one of the two or more liquid parts is a degassed liquid.

28. The obturation material of claim 27, wherein each of the degas sed liquid parts has a percent oxygen content reduction of at least 10%.

29. The obturation material of any one of claims 25 to 28, wherein the free radical initiator comprises a light initiator, chemical initiator, thermal initiator, or a combination thereof.

30. The obturation material of any one of claims 25 to 28, wherein the free radical initiator comprises a light initiator and a thermal initiator.

31. The obturation material of any one of claims 25 to 30, wherein the obturation material is curable upon exposure to human body temperature, and the obturation material has a Shore A hardness of at least 15 when cured.

32. The obturation material of any one of claims 25 to 31, wherein the multi-arm poly(ethylene glycol) acrylate and the multi-arm poly(ethylene glycol) acrylamide are selected from the group consisting of 3-arm PEG acrylate, 3-arm PEG methacrylate, 3-arm poly(ethylene glycol) acrylamide, 3-arm poly(ethylene glycol) triacrylamide, and 4-arm poly(ethylene glycol) acrylamide, or a combination thereof.

33. The obturation material of any one of claims 25 to 31, wherein the acrylate functionalized polyether, the acrylamide functionalized polyether and the methacrylamide functionalized polyether are selected from the group consisting of a Jeffamine® ED-900 acrylate, a Jeffamine® ED-900 acrylamide, a Jeffamine® ED-900 methacrylamide, a Jeffamine® ED 2003 acrylate, a Jeffamine® ED 2003 acrylamide, and a Jeffamine® ED 2003 methacrylamide, or a combination thereof.

34. The obturation material of claim 33, wherein the acrylate functionalized polyether and the acrylamide functionalized polyether are selected from the group consisting of Jeffamine® ED-900 diacrylate, Jeffamine® ED-900 triacrylamide, Jeffamine® ED 2003 diacrylate, and Jeffamine® ED 2003 triacrylamide, or a combination thereof.

35. The obturation material of any one of claims 25 to 34, wherein the radiopaque material comprises sodium diatrizoate hydrate or 5-acrylamido-2,4,6 triiodo isophthalic acid.

36. The obturation material of any one of claims 25 to 35, comprising a cross-linker comprises N,N′-methylenebis(acrylamide) (MB AA), triethylene glycol dimethacrylate (TEGDMA), or a combination thereof.

37. The obturation material of any one of claims 25 to 36, where the obturation material has a hardness value of at least 40 Shore A within 40 seconds of exposure to light energy.

38. A method of preparing a hydrogel comprising forming a reaction mixture comprising the curable mixture or obturation material of any one of claims 1 to 37, wherein the reaction mixture forms the hydrogel upon exposure to human body temperature for a period of time effective to cure the curable mixture.

39. The method of claim 38, further comprising degassing the reaction mixture prior to delivering the reaction mixture to a tooth inside the human body.

40. A method of filling a tooth, comprising:
identifying a tooth having a cavity in need of filling;
positioning the curable mixture or obturation material of any one of claims 1 to 37 within the cavity; and curing the curable mixture or obturation material within the cavity.

41. A method of filling a root canal, comprising:
identifying a tooth having a root canal in need of filling;
positioning the curable mixture or obturation material of any one of claims 1 to 37 within the root canal; and
curing the curable mixture or obturation material within the root canal.

42. A method of filling a root canal with a hydrogel polymer, comprising:
(a) identifying a tooth having a root canal in need of filling;
(b) positioning an aqueous curable mixture or obturation material of any of claims 1 to 37 within a handpiece, comprising delivering the curable mixture or obturation material to the handpiece in two liquid parts;
(c) forming a liquid jet within the handpiece and using the liquid jet to deliver the two parts;
(d) partially curing the curable mixture or obturation material within the root canal with light energy; and
(e) exposing the partially cured mixture or obturation material within the root canal to heat to form a cured hydrogel polymer within the root canal.

43. A curable mixture of ingredients, comprising:
(a) 14 wt. % to 65 wt. % poly(ethylene glycol) diacrylate;
(b) 15 wt. % to 45 wt. % of 5-acrylamido-2,4,6-triiodo isophthalic acid;
(c) 0.2 wt. % to 6 wt. % a free-radical polymerization initiator; and
(d) 20 wt. % to 60 wt. % of an aqueous carrier,
wherein the ingredients are selected to provide the curable mixture with properties suitable for use as a tooth filling material after curing.

44. The curable mixture of claim 43, where the free-radical polymerization initiator is a chemical initiator.

45. The curable mixture of claim 43 or 44, comprising 0.3 wt. % to 1.0 wt. % of a second free-radical polymerization initiator that is a light initiator.

46. A curable mixture of ingredients, comprising:
(a) 14 wt. % to 65 wt. % poly(ethylene glycol) diacrylate;
(b) 0.2 wt. % to 6 wt. % potassium persulfate;
(c) 0.3 wt. % to 1.0 wt. % 1-phenyl-1,2-propanedione (PPD);
(d) 15 wt. % to 50 wt. % of 5-acrylamido-2,4,6-triiodo isophthalic acid; and
(e) 20 wt. % to 60 wt. % of an aqueous carrier,
wherein the ingredients are selected to provide properties suitable for use as a tooth filling material after curing.

47. The curable mixture of any of claims 43 through 46, comprising
(a) 15 wt. % to 55 wt. % poly(ethylene glycol) diacrylate; and
(b) 20 wt. % to 40 wt. % of 5-acrylamido-2,4,6-triiodo isophthalic acid.

48. The curable mixture of any of claims 43 through 47, comprising from 30 wt % to wt % aqueous carrier.

49. The curable mixture of any of claims 43 through 47, comprising from 40 wt % to wt % aqueous carrier.

50. The curable mixture of any of claims 43 through 49, further comprising a quaternary ammonium salt.

51. The curable mixture of any of claims 43 through 49, further comprising from 0.2 wt % to 5 wt % [2-(acryloyloxy)ethyl]trimethyl-ammonium chloride (EGAA).

52. The curable mixture of any of claims 43 through 51, comprising a co-initiator selected from N-phenylglycine, 2-pyrrolidinone, dimethylaminoethyl acrylate (DMAEA), triethanolamine (TEOA), 1-vinyl-2-pyrrolidone and L-arginine.

53. The curable mixture of any of claims 43 through 52, further comprising from 0.2 wt % to 5 wt % of an acrylamide-based monomer.

54. The curable mixture of any of claims 43 through 52, further comprising an 8-arm poly(ethylene glycol) acrylate crosslinker.

55. The curable mixture of any of claims 43 through 54, further comprising triethanolamine (TEOA).

56. An obturation material for use as a radiopaque tooth filling after curing, the obturation material, provided as two or more liquid parts, comprising:
(a) 15 wt. % to 65 wt. % poly(ethylene glycol) diacrylate;
(b) 15 wt. % to 45 wt. % of 5-acrylamido-2,4,6-triiodo isophthalic acid;
(c) 0.2 wt. % to 6 wt. % a first free-radical polymerization initiator;
(d) 0.3 wt. % to 1.0 wt. % a second free-radical polymerization initiator that is a light initiator; and
(e) 20 wt. % to 60 wt. % of an aqueous carrier,
wherein at least one liquid part comprising (a) through (d) has a viscosity less than 60 cP (at 25° C.); wherein(a), (b), (c), and (d) are selected to form a curable mixture;
and wherein the obturation material is curable in a tooth by a polymerization of ingredient (a) that is initiated by ingredients (c) and (d).

57. The obturation material of claim 56, comprising
(a) 15 wt. % to 60 wt. % poly(ethylene glycol) diacrylate;
(b) 15 wt. % to 45 wt. % of 5-acrylamido-2,4,6-triiodo isophthalic acid
(c) 0.2 wt. % to 6 wt. % potassium persulfate;
(d) 0.3 wt. % to 1.0 wt. % 1-phenyl-1,2-propanedione (PPD); and
(e) 30 wt. % to 60 wt. % of an aqueous carrier.

58. The obturation material of claim 56 or 57, wherein at least one liquid part has a viscosity less than 20 cP (25° C.).

59. The obturation material of any one of claims 56 through 58, curable to a Shore A hardness of at least 15 upon light curing with an exposure of 10 seconds.

60. A curable mixture of ingredients, comprising:
(a) 20 wt. % to 65 wt % 3-arm poly(ethylene glycol) acrylate;
(b) 15 wt % to 30 wt. % of 5-acrylamido-2,4,6-triiodo isophthalic acid;
(c) at least one free-radical polymerization initiator in an amount of 0.2 wt. % to 1.5 wt %; and
(d) 20 wt % to 60 wt % of an aqueous carrier.

61. The curable mixture of claim 60, comprising 0.3 wt % to 1.0 wt % of a second free-radical polymerization initiator, wherein at least one is a light initiator.

62. The curable mixture of claim 60, comprising 0.2 wt % to 6% of a co-initiator.

63. The curable mixture of claim 60, comprising 0.2 wt. % to 1.5 wt % potassium persulfate.

64. The curable mixture of claim 60 or 63, comprising 0.3 wt % to 1.0 wt % 1-phenyl-1,2-propanedione (PPD).

65. The curable mixture of any one of claims 60 through 64, comprising 0.1 wt % to 3 wt % triethanolamine (TEOA).

66. An obturation material for use as a radiopaque tooth filling after curing, the obturation material, provided as two or more liquid parts, comprising:
(a) 20 wt. % to 65 wt % 3-arm poly(ethylene glycol) acrylate;
(b) 15 wt % to 30 wt. % of 5-acrylamido-2,4,6-triiodo isophthalic acid;
(c) 0.2 wt. % to 1.5 wt % of a first free-radical polymerization initiator;
(d) optionally, 0.3 wt % to 1.0 wt % of a second free-radical polymerization initiator that is a light initiator;
(e) optionally, 0.2 wt % to 6% of a co-initiator; and
(f) 20 wt % to 60 wt % of an aqueous carrier,
wherein at least one liquid part has a viscosity less than 60 cP (at 25° C.), wherein
(a), (b), (c), (d) and (e) are selected to form a curable mixture, and wherein the obturation material is curable in a tooth by a polymerization of ingredient (a) that is initiated by ingredient (c) and/or (d).

67. The obturation material of claim 66, wherein at least one liquid part has a viscosity less than 20 cP (25° C.).

68. The obturation material of claim 66 or 67, curable to a Shore A hardness of at least 15 upon light curing with an exposure of 10 seconds.

69. A method of filling a root canal with the hydrogel polymer of claim, comprising:
(a) identifying a tooth having a root canal in need of filling;
(b) positioning an aqueous curable mixture or obturation material of any of claims 43 through 68, within a handpiece, comprising delivering the curable mixture or obturation material to the handpiece in two liquid parts;
(c) forming a liquid jet within the handpiece and using the liquid jet to deliver the two parts;
(d) partially curing the curable mixture or obturation material within the root canal with light energy; and
(e) exposing the partially cured mixture or obturation material within the root canal to heat to form a cured hydrogel polymer within the root canal.

70. A curable mixture of ingredients, comprising:
(a) 15 wt. % to 65 wt. % water soluble acrylamide-based monomer;
(b) 15 wt. % to 45 wt. % water soluble ionic or non-ionic iodinated X-ray contrast acrylate monomer;
(c) 0.2 wt. % to 6 wt. % a free-radical polymerization initiator; and
(d) 15 wt. % to 60 wt. % of an aqueous carrier,
wherein the ingredients are selected to provide the curable mixture with properties suitable for use as a tooth filling after curing of the curable mixture to form a cured mixture.

71. The curable mixture of claim 70, comprising:
(a) 15 wt. % to 60 wt. % water soluble acrylamide-based monomer;
(b) 15 wt. % to 45 wt. % 5-acrylamido-2,4,6-triiodo isophthalic acid;
(c) 0.2 wt. % to 6 wt. % of a free-radical polymerization initiator;
(d) 0.1 wt % to 0.3 wt % of a quaternary ammonium salt; and
(d) 15 wt. % to 60 wt. % of an aqueous carrier.

72. The curable mixture of claim 71, wherein the acrylamide-based monomer comprises acrylamide, diacrylamide, triacrylamide, methacrylamide, or a combination thereof.

73. The curable mixture of claim 71, wherein the acrylamide-based monomer comprises poly(ethylene glycol) acrylamide, poly(ethylene glycol) diacrylamide, poly(ethylene glycol) triacrylamide, or a combination thereof.

74. The curable mixture of claim 71, wherein the acrylamide-based monomer comprises a multi-arm poly(ethylene glycol) acrylamide-based monomer.

75. The curable mixture of claim 71, wherein the acrylamide-based monomer comprises a 3-arm poly(ethylene glycol) acrylamide-based monomer, 4-arm poly(ethylene glycol) acrylamide-based monomer, 8-arm poly(ethylene glycol) acrylamide-based monomer, or a combination thereof.

76. The curable mixture of claim 71, wherein the acrylamide-based monomer comprises N,N′-(dimethyl)-ethylenebisacrylamide, bis[2-(2-methyl-acrylamino)-ethoxycarbonyl]-hexamethylenediamine, or N,N′-diethyl-1,3-propylene-bis-acrylamide, or a combination thereof.

77. An obturation material for use as a radiopaque tooth filling after curing, the obturation material, provided as two or more liquid parts, comprising:
(a) 15 wt. % to 65 wt. % water soluble acrylamide-based monomer;
(b) 15 wt % to 30 wt. % of 5-acrylamido-2,4,6-triiodo isophthalic acid;
(c) 0.2 wt. % to 1.5 wt % of a free-radical polymerization initiator;
(d) optionally, 0.3 wt % to 1.0 wt % of a second free-radical polymerization initiator that is a light initiator;
(e) optionally, 0.2 wt % to 6% of a co-initiator; and
(f) 20 wt % to 60 wt % of an aqueous carrier,
wherein at least one liquid part has a viscosity less than 60 cP (at 25° C.), wherein (a), (b), (c), (d) and (e) are selected to form a curable mixture, and wherein the obturation material is curable in a tooth by a polymerization of ingredient (a) that is initiated by ingredient (c) and/or (d).

78. A method of filling a root canal with the hydrogel polymer of claim, comprising:
(a) identifying a tooth having a root canal in need of filling;
(b) positioning an aqueous curable mixture or obturation material of any of claims 71 through 77, within a handpiece, comprising delivering the curable mixture or obturation material to the handpiece in two liquid parts;
(c) forming a liquid collimated jet within the handpiece and using the liquid jet to deliver the two parts;
(d) at least partially curing the curable mixture or obturation material within the root canal with light energy; and
(e) allowing the partially cured mixture or obturation material within the root canal to further chemically cure to form a cured hydrogel polymer within the root canal;
wherein at least one liquid part has a viscosity less than 60 cP (at 25° C.).

79. A curable mixture of ingredients, comprising:
(a) 15 wt. % to 65 wt. % water soluble acrylate-based monomer, acrylamide-based monomer, or a combination thereof;
(b) 15 wt. % to 45 wt. % water soluble iodinated X-ray contrast acrylate monomer;
(c) 0.2 wt. % to 6 wt. % a free-radical polymerization initiator; and
(d) 15 wt. % to 60 wt. % of an aqueous carrier,
wherein the ingredients are selected to provide properties suitable for use as a tooth filling material after curing.

80. The curable mixture of claim 79, wherein the iodinated X-ray contrast acrylate monomer is ionic.

81. The curable mixture of claim 79, wherein the iodinated X-ray contrast acrylate monomer is non-ionic.

82. The curable mixture of claim 79, wherein the X-ray contrast acrylate monomer comprises 5-acrylamido-2,4,6-triiodo isophthalic acid, 2,4,6-Triiodo-5-[(1-oxo-2-propen-1-yl)amino]-1,3-benzenedicarboxylic acid, 5-acryloylamino-N,N′-bis-[(2-hydroxy-1,1-bis-hydroxymethyl-ethylcarbamoyl)-methyl]-2,4,6-triiodoisophthalamide, 5-acryloylamino-N-(2,3-dihydroxypropyl)-N′-(2-hydroxy-1,1-bis-hydroxymethylethyl)-2,4,6-triiodoisophthalamide, or a combination thereof.

83. An obturation material, for use as a tooth filling material after curing, comprising
a curable mixture provided as two or more liquid parts, comprising:
(a) a water-soluble acrylate-based monomer, a water-soluble acrylamide-based monomer, a water-soluble chelating monomer, or a mixture thereof;
(b) calcium silicate dispersed in a non-aqueous carrier;
(c) a free-radical polymerization initiator;
(d) a water-soluble radiopaque material; and
(e) a low viscosity liquid;
wherein components (a) and (b) are selected to provide properties suitable for use as a tooth filling material after curing to form a cured mixture by polymerization of ingredient (a) that is initiated by ingredient (c).

84. The obturation material of claim 83, wherein (a) comprises poly(ethylene glycol) diacrylate.

85. The obturation material of claim 83, wherein (d) comprises 5-acrylamido-2,4,6-triiodo isophthalic acid, 2,4,6-Triiodo-5-[(1-oxo-2-propen-1-yl)amino]-1,3-benzenedicarboxylic acid, 5-acryloylamino-N,N′-bis-[(2-hydroxy-1,1-bis-hydroxymethyl-ethylcarbamoyl)-methyl]-2,4,6-triiodoisophthalamide,5-acryloylamino-N-(2,3-dihydroxypropyl)-N′-(2-hydroxy-1,1-bis-hydroxymethylethyl)-2,4,6-triiodoisophthalamide, or a combination thereof.

86. The obturation material of claim 83 further comprising a crosslinker.

87. The obturation material of claim 83 further comprising an 8-arm poly(ethylene glycol) diacrylate.

88. The obturation material of claim 83 further comprising MBAA.

89. The obturation material of any of claims 83 through 88 wherein component (a) comprises between about 15 wt % to 40 wt % of at least one water-soluble acrylate-based monomer, water-soluble acrylamide-based monomer, or a combination thereof.

90. The obturation material of any of claims 83 through 89 wherein (a) is provided as an aqueous solution.

91. The obturation material of claim 90, comprising an aqueous solution further comprising from about 0.1 wt % to about 5 wt % of a base selected from calcium hydroxide, lithium hydroxide or sodium hydroxide.

92. The obturation material of any of claims 83 through 91, wherein (b) comprises about 10 wt % to 60 wt % of calcium silicate, based on the total weight of the components of (b).

93. The obturation material of any of claims 83 through 92, wherein (b) comprises tricalcium silicate.

94. The obturation material of any of claims 83 through 91, wherein the calcium silicate consists essentially of tricalcium silicate.

95. The obturation material of any of claims 83 through 94, wherein (b) further comprises propylene glycol or poly(ethylene glycol) diacrylate as a non-aqueous carrier liquid.

96. The obturation material of claim 94, comprising less than 40 wt % tricalcium silicate based on the total weight of the curable material.

97. The obturation material of claim 94, comprising less than 30 wt % tricalcium silicate based on the total weight of the curable material.

98. The obturation material of claim 94, comprising less than 25 wt % tricalcium silicate based on the total weight of the curable material.

99. The obturation material of claim 94, comprising less than 20 wt % tricalcium silicate based on the total weight of the curable material.

100. The obturation material of claim 94, comprising less than 15 wt % tricalcium silicate based on the total weight of the curable material.

101. The obturation material of any of claims 83 through 100, wherein a first liquid part comprises from about 3 wt % to 20 wt % of a water-soluble radiopaque material and from about 80 wt % to 97 wt % of the water soluble monomer of (a), a second liquid part comprises water, and a third part comprises calcium silicate and a non-aqueous carrier liquid.

102. The obturation material of any of claims 83 through 101, further comprising potassium persulfate.

103. The obturation material of claim 83, wherein the low viscosity liquid has a viscosity less than 60 cP (at 25° C.).

104. The obturation material of any one of claims 83 through 103, wherein the curable mixture is provided as three parts.
